(19) United States
(12) Patent Application Publication
(10) Pub. No.: US 2011/0212434 A1
(43) Pub. Date: Sep. 1, 2011

(54) NOVEL HIV-BASED RECOMBINANT VIRAL CLONES AND USE THEREOF IN ANALYTICAL METHODS

(76) Inventors: Jose ALCAMI PERTEJO, Madrid (ES); Javier Garcia Perez, Madrid (ES); Sonsoles Sanchez Palomino, Barcelona (ES); Nuria Gonzalez Fernandez, Madrid (ES)

(21) Appl. No.: 13/015,842

(22) Filed: Jan. 28, 2011

Related U.S. Application Data
(63) Continuation-in-part of application No. 11/596,259, filed on May 14, 2007.

(30) Foreign Application Priority Data
May 10, 2004 (ES) P2004-01116

(51) Int. Cl. C12Q 1/70; C12N 7/01

(52) U.S. Cl. 435/5; 435/235.1

(57) ABSTRACT

The present invention refers to HIV-based recombinant viral clones that possess the general structure represented in FIG. 8 and are the result of the following genetic manipulations:
deletion of HIV fragments (for example, Nef gene) without losing infective capacity,
insertion of a non-expressed gene in human cells,
insertion of LacZ gene,
introduction of restriction sites for extracting DNA fragments of matrix provirus and substituting them for genes from patients to assess.

The present invention also refers to the application of these clones in analytical methods related to AIDS.

### TECHNICAL FIELD OF THE INVENTION

Within the broad field of research being conducted into AIDS and more specifically into the development of new families of drugs, the present invention focuses on the generation of certain new recombinant viral clones based on the genome of Human Immunodeficiency Virus (HIV) intended for being advantageously used in sensitivity tests to drugs, detection assays for neutralising antibodies, study of tropism and viral replicative capacity and methods of screening and characterisation of compounds with antiviral activity, etc.

### STATE OF THE ART PRIOR TO THE INVENTION

In the last five years the clinical evolution of patients infected with HIV has improved spectacularly thanks to the introduction of new families of antiretroviral drugs (Havlir and Lange, 1998), and as a consequence there has been a fall in the number of cases of AIDS, of the incidence of opportunistic infections and of mortality as a result of this disease.

Nevertheless, the successes achieved with those drugs have regrettably not made it possible to eradicate the disease since, in spite of the decrease in the plasmatic viral load to undetectable levels, viral replication persists at a low level in lymphoid organs (Chun et al., 1997; Finzi et al., 1997; Wong et al., 1997). Moreover, the proviral load, which reflects the pool of lymphocytes infected by HIV, does not decrease with antiretroviral treatment or it does so very slowly (Sharkey et al., 2000; Ramratnam et al., 2000). Finally, the suspension of antiretroviral medication leads to a rapid upturn in the viral load to base levels, even in patients that were found to be in apparently complete virological suppression (<5 copies of RNA/ml) for two years (García et al., 1999). All this data suggests that the outlook for the eradication of AIDS with currently available medicines seems unlikely (Ho, 1998; Wein et al., 1998; Zhang et al., 1999; Furtado et al., 1999; Pomerantz, 1999). This possibility of eradication entails the development in the medium term of viruses resistant to the antiretroviral drugs used in each patient.

In this situation, a series of strategies against this disease continues to be underway, which can be summarised in the following points:
Development of new drugs, and especially of new families of compounds with different targets from those currently considered by antiretroviral drugs.
Development of therapeutic and preventive vaccines.
Development of immunotherapy strategies aimed at strengthening the patient's immunological system.

Concomitant with the development of these strategies for fighting the disease, it is essential to develop analytical methods and techniques for evaluating these new approaches: models for determination of resistances to antiretrovirals, biological characterisation of qualitative aspects of the biology of the virus and development of models for the generation of platforms for screening and characterisation of the antiviral activity of the new compounds. In the following paragraphs, reference will be made to some of the analytical methods being used at present, and on which this invention has a special impact on account of its advantageous contributions.

Systems for Determination of Phenotypic Resistances to Antiretroviral Drugs.

The determination of phenotypic resistances is not done routinely in patients with HIV infection displaying virological failure, due to their extreme laboriousness and high cost. These tests on phenotypic resistances are habitually done by a method selected from among one of the following two groups of systems:
a. Classical systems: In a first step, these consist of the isolation of the HIV starting from cultures of the patient's lymphocytes and, in a second step, infection of the target cells in the presence of different antiretrovirals in order to determine the inhibition concentration of the drugs (IC50) on a specific isolate. These systems are terribly expensive, lengthy, tedious and they require bio-security systems that are within the reach of very few virology laboratories (Richman et al., 1993, Nagy et al.; 1994).
b: Systems based on genetic recombination techniques. In this technology, the sequences of the pol gene are amplified on the basis of the patient's plasma and transfected together with the provirus selected in those sequences, in cell lines. By means of in vivo ligation reactions inside these cells, a virus carrying the Reverse Transcriptase and Protease sequences from the patient's virus is recombined. The recombinant viral progeny that is generated is used for evaluating the IC50 in the infection of target cells. There exist different variants of this technology in terms of the sequences and steps for amplification, target cells and use of markers (Boucher et al., 1996; Hertogs et al., 1998; Ruiz et al., 1998; Little et al., 1999; Borden et al., 1999). In spite of these developments which simplify the classical systems, testing techniques for viral recombination have limitations such as the low in vivo recombination rates, and it is still expensive and laborious.

Owing to its complexity and difficulties of standardisation, tests on phenotypic resistance to antiretroviral drugs are in practice available in a small number of laboratories and are essentially used for diagnostic purposes.

So, there exists a need for new techniques, simpler and more accessible, which would permit these determinations to be made in any laboratory, quickly, simply and economically.

Systems for the Determination of the Replicative Capacity of HIV.

Among the qualitative characteristics to be found among the existing different isolates of HIV is “replicative capacity” or viral “fitness” (Ruiz Jarabo et al., 2002; Domingo, et al., 2001). Viral fitness is the final result of a multiple set of characteristics of the virus in the process of adaptation to its host. Nevertheless, in some situations, it has been seen that a diminished viral fitness is associated with the clinical evolution of the disease (Tersmette et al., 1995; Learmont et al., 1995). In particular, in a high percentage of long-term surviving patients it is extremely difficult to isolate their viruses in culture owing to their low replicative capacity (Cao et al., 1995; Pantaleo, et al., 1995; Michael et al., 1995). Perhaps of greater clinical relevance is the fact that viruses from multiresistant patients seem to replicate with a lower capacity (Mammano et al., 2000; Martinez-Picado et al.; 2000; Nijhuis et al., 2001; Spira et al., 2003).

The systems for determination of viral fitness are based on competition studies in culture between a wild virus and a virus displaying different mutations (Yuste et al., 1999; Iglesias et al., 2002). A competition studies refer to different techniques that compare the capacity of replication or growth in culture between different viral strains. For example, a competition studies between a wild virus and a virus displaying different mutations of resistance to antiretroviral drugs. These methods require prolonged cultures and are therefore very laborious, expensive and difficult to standardise. The use of recombinant viruses for determining viral fitness has only recently been proposed (Deeka et al., 2001; Barbour et al., 2002) though this technique has not been properly standardised at the present time. With the aim of being able to assess in a precise way the replicative capacity of the virus, it is essential to be able to have techniques that are simple, reliable, accessible and rapid.

Systems for the Detection of the Presence of Neutralising Antibodies as an Efficacy Response Parameter to Experimental Vaccines and Immunomodulator Treatments.

Infection by a virus induces a dual specific immune response in the host: activation of cytotoxic lymphocytes and production of antibodies (McMichael A., 2001; Burton D R., 2002). Of the latter, only those antibodies which block the entry of the virus in the target cell by various mechanisms possess efficacy in controlling the infection. This type of antibody is said to be “neutralising” and the importance of their role in HIV infection has been demonstrated by different works in recent years (Burton D R, 2002; Moore J and Burton D R, 1999).

The measurement of neutralising antibodies is important in a series of clinical situations since it has been shown that their presence is associated with a good prognosis for the infection (Cao et al., 1995; Lathey et al., 1997; Pilgrim et al., 1997; Lomig-Price, et al., 1998). Nevertheless, the greatest application of neutralising antibodies in the next few years will be taking place in the evaluation of new vaccines against HIV. There currently exist more than 50 preparations produced under GMP rules and 35 in phase I and II (McMichael A J and Hanke T, 2003). In evaluating the efficacy of these preparations, the detection of neutralising antibodies will, together with cytotoxic activity against HIV, constitute the two parameters which will decide whether the preparation passes on to more advanced clinical study phases (Poignard et al., 1999; Moore J P and Burton D R.; 1999; McMichael A J and Rowland Jones S L, 2001). When indicating the cytotoxic activity against HIV, it is referred the capacity of host lymphocytes to recognize and destroy in a specific manner those cells infected by HIV-1.

The neutralisation tests or tests for detection of neutralising antibodies are conducted by measuring the inhibition of cellular lysis by HIV in in vitro infection systems (Sattentau Q., 1996; Langlois et al., 1998).

This model has two important drawbacks:
a. An indirect effect of the viral replication is measured: that of cell destruction, but the replication of HIV is not measured directly.
b. The inhibition of a laboratory strain is analysed which means that antibodies against the specific virus of the patient are not detected, an aspect which can affect the characterisation of a specific response of the host.

Immunomodulator treatments refers to all medical interventions using drugs, growth factors, antigens or antibodies that increase the strength and capacity of the immune system to respond to infectious agents or tumours.

Other techniques have been proposed based on microscopy or cytometry of infected cells but they entail a complexity that does not make them viable as routine tests (Haussmann et al., 1987; Mascola et el., 2002). The technique of infection inhibition by means of recombinant viruses has recently been introduced for analysing the neutralising capacity of serums in different experimental approaches (Kolchinski et al., 2001) and in clinical samples (Wei et al., 2003; Richman et al., 2003). It is therefore essential to develop new techniques for solving these two major drawbacks, permitting direct analysis of viral replication and its inhibition by the patient's antibodies, having high sensitivity and reliability and which can be conducted simply, quickly and economically.

Recombinant viruses are defined as viral genomes in which a gene or a fragment of a gene in the original virus is replaced by molecular cloning with a genetic sequence from the same gene of fragment of a gene that has been amplified from cells or plasma of a given patient or other viral isolates. The resulting recombinant virus represents a chimera between the backbone of the original virus and a gene or genetic fragment. The recombinant virus generated displays the functional properties of the viral isolate from which the gene or fragment of gene has been cloned.

Systems for the Characterisation of Viral Tropism in HIV Infection.

As well as the quantitative aspects of viral replication expressed by the plasmatic viral load, the different variants of HIV have a series of biological characteristics which characterise their pathogenicity. Among these, viral tropism, or the capacity of HIV to enter the cell via various receptors, is one of the most important viral characteristics (Weiss R A, 1996; Oberlin et al., 1997; Dorantz et al., 1996; Glushakova et al., 1998).

The existence of two larger receptors of HIV, known as CCR5 and CXCR4 (Loetscher et al., 2000) means that the different viral variants are classified into three categories: R5, X4 and R5X4 in line with their capacity to enter the cell by one of the two receptors exclusively or both receptors (Berger et al., 1998).

The measurement of viral tropism is not normally done as a diagnostic test but it does represent a highly useful parameter in certain areas of research. Nevertheless, the introduction of specific drugs into the entry having as their target one of the two receptors, CCR5 or CXCR4, means that a characterisation of the viral tropism of the patient before commencing treatment with regard to one of these targets can very likely be expected in the future (Lazzarin et al., 2003; Este J A, 2003; Zaitseva et al, 2003).

So, there exists a need to have systems permitting the characterisation of viral tropism in HIV infection in a patient, by means of techniques that are simple and accessible to any analysis laboratory, systems which are so far unavailable.

Experimental Models Permitting Rapid Screening of Compounds with Potential Antiviral Activity.

Current treatments do not permit a cure of HIV infection and so the development of new drugs is a priority in the context of research into AIDS (De Clercq et al., 2002). In essence, two sources of new drugs exist: derivatives of natural products, essentially coming from the plant kingdom, or those generated by combinatory chemistry starting from computer models or crystalline structures of the target molecule (Chu and Cutler, 1992; Jung et al., 2000; Knowles et al., 2003; Rudin et el., 2003; Agrafiotis et al., 2002).

In both cases, the molecule and its derivatives have to be characterised in terms of their toxicity and antiviral activity in a series of models which have to be robotisable in order to permit efficient screening since thousands of compounds have to be tested. There exist different systems currently used from the classical ones in which protection against the cytopathic effect of a reference virus is measured (Pauwels et al., 1987) or specific ones which analyse a certain target by means of biochemical tests (Hazuda et al., 2000; Cherepanov et al., 1997; Walters et al., 2003).

Nevertheless, there continues to exist a demand for screening systems which permit the development of robotisable models with which screening tests can be carried out on thousands of compounds in a way that is faster, more reliable, safer and cheaper (Federsel et al., 2003; Bleicher et al., 2003).

So, in view of the situation described above, the applicant has directed his investigative efforts towards the search for new recombinant viral clones, whose creation, identification and applications have allowed him to conclude the present invention, which represents a great advance in solving the problems and drawbacks mentioned above, as will easily be deduced from a thorough reading of the rest of this descriptive specification.

Provided below is a list of the complete bibliographical references that have been cited above in the foregoing paragraphs.
Agrafiotis D K et al., Combinatorial informatics in the post-genomic ERA. Nat rev Drug Discov. 2002, 1:227.
Barbour J D, Wrin T, Grant R M, Martin J N, Segal M R, Petropouls C J, Deeks S G. Evolution of phenotypic drug susceptibility and viral replication capacity during long-term virologic failure of protease inhibitor therapy in human immunodeficiency virus-infected adults. J Virol, 20002; 76: 11104-12.
Berger E A, Doms R W, Fenyo E M, Korber B T, Littman D R, Moore J P, Sattentau Q J, Schuitemaker H, Sodroski J, Weiss R A. A new classification for HIV-1. Nature. 1998; 391. 240.
Bleicher K H. Hit and lead generation: beyond high-throughput screening. Nat Rev Drug Discov. 2003; 2:369.
Borden D, Hurley A, Zhang, L et al., HIV-1 drug resistance in newly infected individuals. JAMA 1999; 262: 1135-41.
Boucher C A, Keulen W, van Bommel T, Nijhuis M, de Jong D, de Jong M D, Schipper P, Back N K. Human immunodeficiency virus type 1 drug susceptibility determination by using recombinant viruses generated from patients tested in a cell-killing assay. Antomicrob Agents Chemother 1996; 40: 2404-9.
Burton D R. Opinion: Antibodies, viruses and vaccines. Nat Rev Immunol. 2002; 2: 706-13.
Cao Y, Qin L, Zhang L, Safrit J, Ho D D. Virologic and immunologic characterisation of long-term survivors of human immunodeficiency virus type 1 infection. N Engl J Med. 1995; 332: 201-8.
Cherepanov. Mode of interaction of G-quartets with the integrase of HIV. Mol. Pharmacol. 1997. 52: 771.
Chu C K and Cutler H G (Eds.). Natural Products as Antiviral Agents. Ed. C. K., (1992), Plenum Press, N.Y.
Chun T W, Carruth L, Finzi D et al. Quantification of latent tissue reservoirs and total body viral load in HIV-1 infection. Nature 1997; 37: 183-8.
Chun T W, Stuyver L, Mizell S B, Ehler L A, Mican J A, Baseler M, Lloyd A L, Nowank M A, Fauci A S. Presence of an inducible HIV-1 latent reservoir during highly active antiretroviral therapy. Proc Natl Acad Sci USA 1997 Nov. 25; 94 (24): 13193-7.
De Clercq E. Strategies in the design of antiviral drugs. Nat Rev Drug Discov. 2002; 1: 13-25.
Deeks S G, Wrin T, Liegler T, Hoh R, Hayden M, Barbour J D, Hellmann N S, Petropoulos C J, McCune J M, Hellerstein N S, Grant R M. Virologic and immunologic consequences of discounting combination antiretroviral drug therapy in HIV-inflected patients with detectable viremia. N Engl J Med. 2001; 344: 472-80.
Domingo E, Mas A, Yuste E, Pariente E, Sierra S, Gutierrez-Riva M, Menendez-Arias L. Virus population dynamics, fitness variations and the control of viral disease: an update. Prog Drug Res. 2001; 57: 77-115.
Dorantz B J, Rucker J, Yi Y, et al. A dual-tropic primary HIV-1 isolate that uses fusin and the beta-chemokine receptors CKR5, CKR3 and CKR2 as fusion coreceptors. Cell 1996; 35: 1149-1158.
Este J A. Virus entry as a target for anti-HIV intervention. Curr Med. Chem. 2003; 10: 1617-32.
Federsel H J. Logistics of process R&D: transforming laboratory methods to manufacturing scale. Nat Rev Drug Discov. 2003; 2: 654.
Finzi D, Hermankova M, Pierson T, et al. Identification of a reservoir for HIV-1 in patients on highly active antiretroviral therapy. Science 1997; 278: 1295-300.
Furtado M, Callaways D S, Phair J P et al. Persistance of HIV transcription in patients receiving combination antiretroviral therapy. N. Engl. J. Med. 1999; 340: 1614-22.
Garcia F, Plana M, Vidal C. et al. Dynamics of viral load rebound and immunological changes after stopping effective antiretroviral therapy. AIDS 1999; 13: f79.86.
Glushakova S, Grivel J C, Fitzgerald W, Sylvester A, Zimmerberg J, Margolis L B. Evidence for HIV-1 phenotype switch as a causal factor in acquired immunodeficiency. Nature Med 1998; 4: 346-348.
Hausmann E H, Gelderblom H E, Clapham P R, Pauli G, Weiss R A. Detection of HIV envelope specific antibodies by immunoelectron microscopy and correlation with antibody, titer and virus neutralizing activity. J Virol Methods. 1987; 16: 125-37.
Havlir D V, Lange J M. New antiretrovirals and new combinations. AIDS 1998; 12 Suppl A:S165-74.
Havlir D V, Marschner I C, Hirsch M S et al. Maintenance antiretroviral therapies in HIV infected patients with undetectable plasma HIV RNA after triple-drug therapy. AIDS Clinical Trials Group Study 343 Team. N Engl J Med 1998; 339: 1261-8.
Hazuda D J et al. Inhibitors of strand transfer that prevent integration and inhibit HIV-1 replication in cells. Science 2000; 287: 646.
Hertogs K, de Bethune M P, Miller V et al. A rapid method for simultaneous detection of phenotypic resistance to inhibitors of protease and reverse transcriptase in recombinant human immunodeficiency virus type 1 isolates from patients treated with antiretroviral drugs. Antimicrob Agents Chemother 1998; 42: 269-76.
Ho D D. Toward HIV eradication or remission: the tasks ahead. Science 1998; 280: 1866-7.
Iglesias-Ussel M D, Casado C, Yuste E, Olivares I, Lopez-Galindez C. In vitro analysis of human immunodeficiency virus type 1 resistance to nevirapine and fitness determination of resistant variants. J Gen Virol. 2002; 83): 93-101.
Jung M. Recent studies on natural products as anti-HIV agents. Curr. Med. Chem. 2000; 649: 125.
Knowles J. A guide to drug discovery: Target selection in drug discovery. Nat Rev Drug Discov. 2003; 2: 63.
Kolchinsky P, Kiprilov E, Sodroski J. Increased neutralization sensitivity of CD4-independent human immunodeficiency virus variants. J Virol. 2001; 75: 2041-50.
Lathey J L, Pratt R D, Spector S A. Appearance of autologous neutralizing antibody correlates with reduction in virus load and phenotype switch during primary infection with human immunodeficiency virus type 1 (Letter). J Infect Dis. 1997; 175: 231-2.
Langlois A J, Matthews T J, Weinhold K J, Chaffee S, Hershfield M, Bolognesi D P. Detection of HIV-1 neutralizing antibodies by a simple, rapid, colorimetric assay. AIDS Ras Hum Retrovisuses. 1988 February; 4(1): 63-9.
Lazzarin A, Clotet B, Cooper D, Reynes J, Arasteh K, Nelson M, Katlama C. Stellbrink H J, Defraissy J F, Lange J, Huson L, DeMasi R, Wat C, Delehanty J, Drobnes C, Salgo M; TORO 2 Study Group. Efficacy of enfuvirtide in patients infected with drug-resistant HIV-1 in Europe and Australia. N Engl J Med. 2003; 348: 2186-95.
Learmont J C, Gezcy A F, Mills J, Ashton L J, Raynes-Greenow C H, Garsia R J, et al. Immunologic and virologic status after 14 to 18 years of infection with an attenuated strain of HIV-1. A report from the Sydney Blood Bank Cohort. N Engl J Med 1999; 340: 1715-22.
Little S J, Daar E S, D'Aquila R T et al. Reduced antiretroviral drug susceptibility among patients with primary HIV infection. JAMA 1999; 282: 1142-49.
Loetscher P, Moser B, Baggiolini M. Chemokines and their receptors in lymphocyte traffic and HIV infection. Adv Immunol. 2000; 74: 127-80.
Loomis-Price L D, Cox J H, Mascola J R, VanCott T C, Michael N L, Fouts T R et al., Correlation between humoral responses to human immunodeficiency virus type 1 envelope and disease progression in early-stage infection. J. Infect Dis. 1998; 178: 1306-16.
Mammano F, Trouplin V, Zennou V, Clavel F. Retracing the evolutionary pathways of human immunodeficiency virus type 1 resistance to protease inhibitors: virus fitness in the absence and in the presence of drug.
J Virol. 2000; 7: 8524-31.
Martinez-Picado J, Savara A V, Shi L, Sutton L, D'Aquila R T. Fitness of human immunodeficiency virus type 1 protease inhibitor-selected single mutants. Virology. 2000 Sep. 30; 275: 318-22.
Mascola J R, Louder M K, Winter C, Prabhakara R, De Rosa S C, Douek D C, Hill B J, Gabuzda D, Roederer M. Human immunodeficiency virus type 1 neutralization measured by flow cytometric quantitation of single-round infection of primary human T cells. J Virol. 2002; 76: 4810-21
McMichael A J and Rowland-Jones S L. Cellular immune responses to HIV. Nature 2001; 410: 980-7.
McMichael A J and Hanke T. HIV vaccines 1998-2003; Nat. Med. 2003; 9: 874-880.
Michael N L, Chang G, d'Arcy L A, Ehrenberg P K, Mariani R, Busch M P, et al. Defective accessory genes in a human immunodeficiency virus type 1-infected long-term survivor lacking recoverable virus. J Virol. 1995; 69: 4228-36.
Moore J P and Burton D R, HIV-1 neutralizing antibodies: how full is the bottle? Nat Med 1999; 5: 142-144
Nagy K, Young M, Baboonian C, Merson J, Whittle P, Oroszlan S. Antiviral activity of human immunodeficiency virus type 1 protease inhibitors in a single cycle of infection: evidence for a role protease in the early phase. J Virol 1994; 68: 757-65.
Nijhuis M, Deeks S and Boucher C. Implications of antiretroviral resistance on viral fitness. Curr. Opinion Inf. Dis. 2001; 14: 23-28
Oberlin E, Amara A, Bachelerie F, et al. The CXc chemokine SDF-1 is the ligand for LESTR/Fusin and prevents infection by a T-cell line adapted HIV-1. Nature 1997; 382: 833-835.
Pantaleo G, Menzo S, Vaccarezza M, Graziosi C, Cohen O J, Demarest J F, et al. Studies in subjects with long-term nonprogressive human immunodeficiency virus infection. N Engl J Med. 1995; 332: 209-16.
Pauwels, R et al. Sensitive and rapid assay on MT-4 cells for detection of antiviral compounds against the AIDS virus, J Virol Methods. 1987. 16: 171-185.
Pilgrim A K, Pantaleo G, Cohen O J, Fink L M, Zhou J Y, Zhou J T, et al. Neutralizing antibody responses to human immunodeficiency virus type 1 in primary infection and long-term-nonprogressive infection. J Infect Dis. 1997; 176: 924-32.
Poignard P. Neutralizing antibodies have limited effects on the control of established HIV-1 infections in vivo. Immunity 1999; 10: 431-438.
Pomeranzt R J Residual HIV-1 disease in the era of HAART. N. Eng. J. Med. 1999; 340: 1625-27.
Ramratnam B, Mitler J E, Zhang L. et al. The decay of the latent reservoir of replication-competent HIV-1 is inversely correlated with the extent of residual viral replication during prolonged antiretroviral therapy. Nat. Med. 2000; 6; 82-85.
Richman D D, Johnson V A, Shirisaka T, O'Brien M C, Mitsuya H. Measurement of susceptibility of HIV-1 to antiviral drug. In: Strober W, Shevach E. eds. Current Protocols in Immunology. New York: Green Publishing Associates, 1993: 12.9.1.
Richman D D, Wrin T, Little S J, Petropoulos C J. Rapid evolution of the neutralizing antibody response to HIV type 1 infection. Proc Natl Acad USA. 2003; 100: 4144-9.
Rudin M et al. Molecular imaging in drug discovery and development. Nat Rev Drug Discov. 2003; 2: 123.
Ruiz L, Nijhuis M, Boucher C, Puig T, et al. Efficacy of adding indinavir to previous reverse transcriptase nucleoside in relation to genotypic and phenotypic resistance development in advanced HIV-1-infected patients. J Acquir Immune Defic Syndr Hum Retrovirol 1998:19:19-28.
Ruiz-Jarabo C M, Arias A, Molina-Paris C, Briones C, Baranowski E, Escarmis C, Domingo E. Duration and fitness dependence of quasispecies memory. J Mol. Biol. 2002; 315: 285-96.
Sattentau Q. Neutralization of HIV-1 by antibody. Curr. Opin. Immunol. 1996; 8: 540-5.
Sharkey M E, Teo I, Greenough T et al. Persistence of episomal HIV-1 infection intermediates in patients on highly active antiretroviral therapy. Nat. Med. 2000; 6: 76-81.
Spira S, Wainberg M A, Loemba H, Turner D, Brenner B G. Impact of Glade diversity on HIV-1 virulence, antiretroviral drug sensitivity and drug resistance. J Antimocrob Chemother. 2003; 51: 229-40.
Tersmette M, Lange J M, de Goede R E, de Wolf F, Eaftink-Schattenkerk J K, Schellekens P T, et al. Association between biological properties of human immunodeficiency virus variants and risk for AIDS and AIDS mortality. Lancet. 1989; 1: 983-5.
Walters W F. Designing screens: how to make your hits a hit. Nat Rev Drug Discov. 2003; 2: 259.
Wei X, Decker J M, Wang S, Hui H, Kappes J C, Wu X, Salazar-Gonzalez J F, Salazar M G, Kilby J M, Saag M S, Komarova N L, Nowak M A, Hahn B H, Kwong P D, Shaw G M. Antibody neutralization and escape by HIV-1. Nature. 2003; 422: 307-12.
Wein L, D'Amato R, Perelson A. Mathematical analysis or antiretroviral therapy aimed at HIV-1 eradication or maintenance of low viral loads. J Theor Biol 1998; 192: 81-98.
Weiss R A. HIV receptors and the pathogenesis of AIDS. Science 1996; 272: 1885-1886.
Wong J K, Hezareh M, Günthard H F, Havlir D V, Ignacio C A, Spina C A and Richman D. recovery of replication competent HIV despite prolonged suppression of plasma viremia. Science 1997; 278: 1291-5.
Yuste E, Sanchez-Palomino S, Casado C, Domingo E, Lopez-Galindez C. Related Articles, Links Drastic fitness loss in human immunodeficiency virus type 1 upon serial bottleneck events. J Virol. 1999; 73; 2745-51.
Zaitseva M, Peden K, Golding H. HIV coreceptors: role of structure, posttranslational modifications, and internalization in viral-cell fusion and as targets for entry inhibitors. Biochim Biophys Acta. 2003; 1614: 51-61.
Zhang I, Ramratnam B, Tenner-Racz K et al. Quantifying residual HIV-1 replication in patients receiving combination antiretroviral therapy. N Engl J Med. 1999; 340: 1605-13.

### DESCRIPTION OF THE INVENTION

The present invention describes a new HIV-based recombinant viral clone which is the result of a series of genetic manipulations made on a specific HIV viral clone, including the amplification of viral fragments from cells or plasma of a given patient by PCR techniques and the subsequent cloning of this fragment in the target recombinant viral clone. The final product so generated includes genes or fragment of genes from the virus of the patients and the genetic backbone of said target recombinant viral clone. The present invention also describes the evaluation of the HIV-based recombinant viral clones subject matter of the present invention, in different systems of analytical determination: determination system for phenotypic resistances to antiretroviral drugs, determination system of the replicative capacity, determination system of viral tropism, phenotypic resistances to fusion inhibitors, system for detection and titration of neutralising antibodies, system for screening compounds and products having potential activity against hiv.

### DETAILED DESCRIPTION OF THE INVENTION

As stated in its title, this invention refers to the generation of new recombinant viral clones based on HIV and their use in analytical methods.

The study of the functional properties of viral strains from patients requires absolutely the viral isolation in culture as a previous step. This process is long, expensive and laborious and is not suitable for diagnosis routine studies of patients in the clinical setting. The invention here described bypass the process of viral culture through the amplification of viral fragments from cells or plasma of a given patient by PCR techniques and the subsequent cloning of this fragment in a recombinant HIV viral clone. The final product so generated includes genes or fragment of genes from the virus of the patients and the genetic backbone of the target recombinant viral clone. The recombinant viral clones of the present invention are the result of a series of genetic manipulations made on an HIV viral clone. The present invention has selected the HIV viral clone NL4.3 (Adachi A. Gendelman H E, Koenig S, Folks T, Willey R, Rabson A, Martin M A. Production of acquired immunodeficiency syndrome-associated retrovirus in human and nonhuman cells transfected with an infectious molecular clone. J. Virol. 1986 August; 59 (2): 284-91)

Within the context of the present invention, an HIV viral clone refers to a fragment of DNA containing the whole genome of the HIV, including the two LTR of the proviral form of the virus. Understanding that when referring to the proviral form of the virus it is referred the proviral DNA as the retrotranscribed full-length HIV genome as it is integrated in an infected cell. Said DNA genome contains two tandem repetitive sequences in the 5′ and 3′ ends that are named LTR (Long Terminal Repeats), three structural genes (gag, pol and env) and five regulatory genes (tat, rev, vif, vpr, vpu and nef).
In addition, within the context of the present invention, a recombinant viral clone based on HIV is defined as a given HIV strain (in our case the NL4.3 HIV viral clone) modificated by means of genetic manipulations including deletion of viral genes, insertion of marker genes, introduction of mutations and substitution of genes or gene fragments from the original clone, with fragments from other clones or viral populations.
Specifically, using this NL4.3 HIV viral clone as template the following genetic manipulations have been performed to generate the recombinant viral clones described in this invention: insertion into the proviral DNA of the NL4.3 HIV viral clone of the marker gene renilla, a non-expressed gene in human cells. This enables the gene to function as a marker of infection, in other words, a cell which expresses renilla indicates that it has been infected and the virus replicates. Within the context of the present invention, the marker gene renilla refers to a gene isolated from sea pansy (Renilla reniformis) that codifies for a 36 kDa protein. This protein displays enzymatic activity and catalyzes a chemical reaction between luciferin and ATP that results in light emission. This gene is not expressed in human cells and therefore enables its use as a marker or reporter gene when it is expressed from different promoters in human cells. In the recombinant viral clones here presented the renilla gene is cloned in the 3′ end of the viral genome and its expression is driven when full-length expression of the HIV genome occurs. Thus, measurement of light emission by cell extracts when luciferine and ATP are added reflects HIV replication.
deletion of HIV fragments of the proviral DNA of the NL4.3 HIV viral clone and subsequent insertion in such positions of LacZ gene which codes for the enzyme Beta-galactosidase, substituting different sequences of the genome in order, on the one hand, to recognise the generation frequency of recombinant viruses and, on the other, to prevent dragging of wild viruses and generating viral populations carrying exclusively viral sequences from a given patient. The LacZ gene encodes one subunit of the Beta-galactosidase from E. coli, a hydrolase enzyme that catalyzes the hydrolysis of β-galactosides into monosaccharides. Beta-galactosidase can be split in two peptides, LacZα and LacZΩ, none of which is active by itself but both spontaneously reassemble into a functional enzyme. This characteristic is used in many cloning vectors to achieve α-complementation in specific laboratory strains of E. coli, where the small LacZα peptide is encoded by the plasmid while the large LacZΩ is encoded in trans by the bacterial chromosome. When DNA fragments are inserted in the vector and production of LacZα is disrupted, the cells exhibit no β-galactosidase activity: this allows the blue/white screening of bacteria that have incorporated the plasmid carrying the complementary LacZ gene. On the other hand, the generation of pure populations represents a major problem in the generation of recombinant viruses. In order to avoid the contamination with sequences coming for the wild type virus used as template, the adopted strategy to prevent dragging of wild viruses consists in the replacement of wild type genetic fragments that will be later cloned from patients with the LacZ gene. The LacZ viral template so generated, lacks the sequences from the wild type virus and is unable to replicate unless sequences from patients become efficiently cloned in the pNL-LacZ vector;
introduction by directed mutagenesis of restriction sites which permit certain DNA fragments of the matrix provirus of the NL4.3 HIV viral clone (such as for example Reverse Transcriptase, Protease, the complete Pol gene, gag, nef or the virus envelope) to be easily “extracted”, so that they can be substituted with genes from isolates coming from patients to be assessed. This “cloning” system and generation of “chimera viruses” permits the characteristics of the different viral proteins of the patients to be studied in a system which presents all the advantages of marker genes.

Within the context of the present invention, the directed mutagenesis refers to genetic manipulation that exchanges a given nucleotide by another. In vitro approaches to site-directed mutagenesis can be grouped generally into three categories: i) methods that restructure fragments of DNA, such as cassette mutagenesis; ii) localized random mutagenesis; and iii) oligonucleotide-directed mutagenesis. Of these methods, oligonucleotide-directed mutagenesis is by far the most commonly used method.

The system of marking with renilla displays many advantages compared to the marker systems most commonly used nowadays and it can be highlighted in particular that:
the detection of renilla render quantification of viral replication 100 fold more sensitive than measurement of reverse transcriptase activity or concentration of viral antigens in culture,
this higher sensitivity in detection of renilla allows the use of microculture techniques (96 or 384 well plates),
this procedure can be used automatically and can even be robotised,
it is a cheap assay, 10 times less expensive than determination of viral replication by viral antigen testing by ELISA,
detection following infection with a virus carrying renilla as a marker is very fast (24 hours) compared to conventional systems for viral replication detection, which require between 5 days and a week of culture.

The HIV-based recombinant viral clones of the present invention are characterised in that they possess the general structure represented in FIG. 8. The present invention refers to an HIV-based recombinant viral clone containing the following elements in 5′ to 3′ direction:

a. LTR or redundant terminal sequences (R) of the proviral DNA of NL4.3 which contains numerous consensus sequences for transcription factors that regulate viral expression. Within the context of the present invention, the LTR or redundant terminal sequences refers to nontranscribed DNA fragments present in both 5′ and 3′ ends of the viral genome in its integrated form. These LTR sequences are redundant and encompass regulatory elements driving viral transcription and mRNA elongation. The regulatory elements of the recombinant viral clones used in the invention are those of the NL4.3 viral clone described in Adachi et al. 1986;
b. gag is the gene which codes the p55 capsid protein formed by 3 protein subunits (MA, CA and NC). Said gag gene can comprise:
b.1) NarI restriction site at position 637 made unique by destruction of the NarI restriction site external to the provirus NL4.3. In this regard, in order to make unique NarI restriction site at position 637 it is necessary the disruption of the NarI restriction site existing at position 13.573 in the NL4.3 plasmid;
c. pol is the gene which codes the viral enzymes needed for the viral replication process: protease (PRO), reverse transcriptase (RT) and integrase, and whose 5′ end overlaps with gag element. If b.1) does not apply, said pol gene can comprise restriction sites generated by directed mutagenesis, said restriction sites being selected from the following group:
c.1) NcoI-AgeI restriction sites at positions 2593 and 3485 respectively,
c.2) ApaI-NcoI restriction sites at positions 2006 and 2593 respectively, and
c.3) ApaI-AgeI restriction sites at positions 2006 and 3485 respectively, or
   if b.1) does apply, said pol gene can comprise:
c.4) KspI restriction site generated by directed mutagenesis at position 4498, and
c.5) ApaI-NcoI restriction sites generated by directed mutagenesis at positions 2006 and 2593 respectively;
   d. vif is the gene that codes the protein Vif, it's 5′ end overlaps with pol element and it's 3′ end overlaps vpr element;
   e. vpr is the gene that codes the protein Vpr and it's 5′ end overlaps vif element;
   f. tat is the gene that codes the protein Tat, it's second exon is contained inside env sequence;
   g. vpu is the gene that codes Vpu;
   h. env is the gene which codes the protein gp160 of the viral envelope, if b.1), c.1), c.2), c.3), c.4) and c.5) do not apply, said env gene can comprise:
h.1) XbaI-NotI restriction sites generated by directed mutagenesis at positions 6114 and 8796 respectively;
   i. rev is the gene that codes the protein Rev, it's second exon is contained inside env sequence;
   j. nef is the gene that codes protein Nef, and is truncated at the bases in positions 8796 and 8887 of the viral genome;
   k. NotI is a restriction site for NotI enzyme, that has been introduced by directed mutagenesis at position 8796 of the viral genome;
   l. XhoI is a restriction site for the XhoI enzyme, in position 8887 of the viral genome;
   m. Renilla is the gene that codes the luciferase reporter protein Renilla, and that has been cloned in restriction sites NotI-XhoI in position 5′ and 3′, respectively;
   n. LTR, whose 5′ end overlaps with the 3′ end of nef element; and
   o. LacZ gene cloned in restriction sites generated by directed mutagenesis, substituting fragments of the viral genome in order to allow further replacement of such sequences with fragments from isolates coming from patients, said restriction sites being selected from the following group:
o.1) ApaI-AgeI restriction sites, in positions 5′ and 3′, respectively, wherein c.3) applies, said LacZ gene is cloned between positions 2006 and 3485, substituting the fragment of pol gene that encode the protease and the reverse transcriptase regions of the viral genome,
o.2) ApaI-NcoI restriction sites in positions 5′ and 3′, respectively, wherein c.2) applies or b.1), c.4) and c.5) applies, said LacZ gene is cloned between positions 2006 and 2593, substituting the fragment of pol gene that encode the protease region of the viral genome,
o.3) NcoI-AgeI restriction sites in positions 5′ and 3′, respectively, wherein c.1) applies, said LacZ gene is cloned between positions 2593 and 3485, substituting the fragment of pol gene that encode the reverse transcriptase region of the viral genome, and
o.4) XbaI-NotI restriction sites in positions 5′ and 3′, respectively, wherein h.1) applies, said LacZ gene is cloned between positions 6114 and 8796, substituting the fragment of env gene of the viral genome.

The presence of all elements a) to o) is indispensable for the correct development of the claimed HIV-based recombinant viral clone.

Summarised below are the stages followed in the generation of the different recombinant HIV-based viral clones of the invention. Given in bold and between brackets is the name of the intermediate and final gene constructions generated:

a) Introduction by directed mutagenesis of the NotI restriction site at the start position 8796) of the nef gene ((IP NL Not).

b) Nef gene deletion between positions 8796 and 8887) (cutting with restriction enzymes NotI and XhoI).

c) Cloning of the renilla gene in NotI/XhoI position (IP HIV NL Ren). See the general structure (FIG. 8).

d) Elimination of the unique NcoI site by means of digestion and filling with Klenow and introduction by directed mutagenesis of another NcoI restriction site in the position corresponding to amino acid 15 of retrotranscriptase, position 2593 of the DNA sequence, (change of glycine for alanine). (IP HIV NL Nco Ren).

e) Cloning in the IP HIV NL Nco Ren vector of the LacZ/beta-galactosidase gene in the position of the RT (IP HIV NL LacZ/rt Ren, fragment 2593-3485) Protease (IP HIV NL LacZ/pr Ren, fragment 2006-2593) or the complete pol gene (IP HIV NL LacZ/pol Ren, fragment 2006-3485) with the aim of increasing the cloning efficacy and preventing dragging of minority populations of the reference virus.

f) Starting from the IP HIV NL LacZ/pr Ren plasmid, destruction of the NarI restriction site external to the provirus in position 13573 of the NL4.3 plasmid by means of directed mutagenesis and introduction of the KspI restriction site in position 4498 by directed mutagenesis (IP HIV NL LacZ/gag-pr Ren).

g) Introduction by directed mutagenesis of the XbaI restriction site at position 6114 in clone IP HIV NL Ren (IP HIV NL Xba Ren).

h) Deletion of the envelope in plasmid IP HIV NL XbaI Ren by means of cutting with the restriction enzymes XbaI and NotI at positions 6114-8796) and cloning in its place of the LacZ gene (IP HIV NL LacZ/env Ren).

i) Generation of the viral clone IP HIV JR Ren cloning the envelope of the JR-CSF clone in the IP HIV NL LacZ/Env Ren plasmid.

The final recombinant HIV-based viral clones thus generated have been deposited in the Spanish Collection of Type Cultures (University of Valencia, Burjassot, Valencia, Spain), in accordance with the rules of the Budapest Treaty on international recognition of deposited microorganisms for the purpose of patent procedure.

The particular structures of those viral clones are given below, indicated between brackets next to their name in the context of the present specification, is the name that has been assigned by the CECT:

### IP HIV NL Ren (CECT 5842)

Recombinant viral clone based on the general structure previously described, characterized in that it possesses unique restriction sites for ApaI and AgeI enzymes introduced at positions 2006 and 3485, respectively, as shown in FIG. 9.

### IP HIV NL LacZ/pol Ren (CECT 5847)

Recombinant viral clone based on the general structure previously described, wherein applies c.3) and o.1) described previously when defining the general structure of the HIV-based recombinant viral clone, characterized in that it possesses the LacZ gene cloned between restriction sites ApaI-AgeI in positions 5′ and 3′, respectively, substituting the fragment of pol gene that codes the protease and the reverse transcriptase, as shown in FIG. 10.

Within the meaning of the present invention, IP HIV NL LacZ/pol Ren (CECT 5847) refers to a recombinant HIV-based viral clone comprising the following elements in 5′ to 3′ direction:

a. LTR or redundant terminal sequences (R) of the proviral DNA of NL4.3;
b. gag is the gene which codes the p55 capsid protein formed by 3 protein subunits (MA, CA and NC);
c. pol is the gene which codes the viral enzymes needed for the viral replication process: protease (PRO), reverse transcriptase (RT) and integrase, and whose 5′ end overlaps with gag element, and comprising ApaI-AgeI restriction sites at positions 2006 and 3485 respectively. This option is described as c.3 when defined previously in the general structure of the HIV-based recombinant viral clone;
d. vif is the gene that codes the protein Vif, it's 5′ end overlaps with pol element and it's 3′ end overlaps vpr element;
e. vpr is the gene that codes the protein Vpr and it's 5′ end overlaps vif element;
f. tat is the gene that codes the protein Tat, it's second exon is contained inside env sequence;
g. vpu is the gene that codes Vpu;
h. env is the gene which codes the protein gp160 of the viral envelope;
i. rev is the gene that codes the protein Rev, it's second exon is contained inside env sequence;
j. nef is the gene that codes protein Nef, and is truncated at the bases in positions 8796 and 8887 of the viral genome;
k. NotI is a restriction site for NotI enzyme, that has been introduced by directed mutagenesis at position 8796 of the viral genome;
l. XhoI is a restriction site for the XhoI enzyme, in position 8887 of the viral genome;
m. Renilla is the gene that codes the luciferase reporter protein Renilla, and that has been cloned in restriction sites NotI-XhoI in position 5′ and 3′, respectively;
n. LTR, whose 5′ end overlaps with the 3′ end of nef element; and
o. LacZ gene cloned in ApaI-AgeI restriction sites, in positions 5′ and 3′, respectively, said LacZ gene is cloned between positions 2006 and 3485, substituting the fragment of pol gene that encode the protease and the reverse transcriptase regions of the viral genome. This option is described as o.1 when defined previously in the general structure of the HIV-based recombinant viral clone.

### IP HIV NL LacZ/pr Ren (CECT 5846)

Recombinant viral clone based on the general structure previously described, wherein applies c.2) and o.2) described previously when defining the general structure of the HIV-based recombinant viral clone, characterized in that it possesses a unique restriction site for NcoI enzyme introduced by directed mutagenesis in position 2593 of the DNA sequence, and the LacZ gene cloned between restriction sites ApaI-NcoI in positions 5′ and 3′, respectively, substituting the fragment of the pol gene that encodes the protease, as shown in FIG. 11.

Within the meaning of the present invention, IP HIV NL LacZ/pr Ren (CECT 5846) refers to a recombinant HIV-based viral clone comprising the following elements in 5′ to 3′ direction:

a. LTR or redundant terminal sequences (R) of the proviral DNA of NL4.3 by directed mutagenesis;
b. gag is the gene which codes the p55 capsid protein formed by 3 protein subunits (MA, CA and NC);
c. pol is the gene which codes the viral enzymes needed for the viral replication process: protease (PRO), reverse transcriptase (RT) and integrase, and whose 5′ end overlaps with gag element, and comprising ApaI-NcoI restriction sites at positions 2006 and 2593 respectively. This option is described as c.2 when defined previously in the general structure of the HIV-based recombinant viral clone;
d. vif is the gene that codes the protein Vif, it's 5′ end overlaps with pol element and it's 3′ end overlaps vpr element;
e. vpr is the gene that codes the protein Vpr and it's 5′ end overlaps vif element;
f. tat is the gene that codes the protein Tat, it's second exon is contained inside env sequence;
g. vpu is the gene that codes Vpu;
h. env is the gene which codes the protein gp160 of the viral envelope;
i. rev is the gene that codes the protein Rev, it's second exon is contained inside env sequence;
j. nef is the gene that codes protein Nef, and is truncated at the bases in positions 8796 and 8887 of the viral genome;
k. NotI is a restriction site for NotI enzyme, that has been introduced by directed mutagenesis at position 8796 of the viral genome;
l. XhoI is a restriction site for the XhoI enzyme, in position 8887 of the viral genome;
m. Renilla is the gene that codes the luciferase reporter protein Renilla, and that has been cloned in restriction sites NotI-XhoI in position 5′ and 3′, respectively;
n. LTR, whose 5′ end overlaps with the 3′ end of nef element; and
o. LacZ gene cloned in restriction sites ApaI-NcoI restriction sites in positions 5′ and 3′, respectively, wherein said LacZ gene is cloned between positions 2006 and 2593, substituting the fragment of pol gene that encode the protease region of the viral genome. This option is described as o.2 when defined previously in the general structure of the HIV-based recombinant viral clone.

### IP HIV NL LacZ/rt Ren (CECT 5845)

Recombinant viral clone based on the general structure described previously, wherein applies c.1) and o.3) described previously when defining the general structure of the HIV-based recombinant viral clone, characterized in that it possesses a unique restriction site for NcoI enzyme that has been introduced by directed mutagenesis in position 2593 of the DNA sequence, and the LacZ gene cloned between restriction sites NcoI-AgeI in position 5′ and 3′, respectively, substituting the fragment of pol gene that encodes the reverse transcriptase (FIG. 12).

Within the meaning of the present invention, IP HIV NL LacZ/rt Ren (CECT 5845) refers to a recombinant HIV-based viral clone comprising the following elements in 5′ to 3′ direction:

a. LTR or redundant terminal sequences (R) of the proviral DNA of NL4.3;
b. gag is the gene which codes the p55 capsid protein formed by 3 protein subunits (MA, CA and NC);
c. pol is the gene which codes the viral enzymes needed for the viral replication process: protease (PRO), reverse transcriptase (RT) and integrase, and whose 5′ end overlaps with gag element, and comprising NcoI-AgeI restriction sites at positions 2593 and 3485 respectively. This option is described as c.1 when defined previously in the general structure of the HIV-based recombinant viral clone;
d. vif is the gene that codes the protein Vif, it's 5′ end overlaps with pol element and it's 3′ end overlaps vpr element;
e. vpr is the gene that codes the protein Vpr and it's 5′ end overlaps vif element;
f. tat is the gene that codes the protein Tat, it's second exon is contained inside env sequence;
g. vpu is the gene that codes Vpu;
h. env is the gene which codes the protein gp160 of the viral envelope;
i. rev is the gene that codes the protein Rev, it's second exon is contained inside env sequence;
j. nef is the gene that codes protein Nef, and is truncated at the bases in positions 8796 and 8887 of the viral genome;
k. NotI is a restriction site for NotI enzyme, that has been introduced by directed mutagenesis at position 8796 of the viral genome;
l. XhoI is a restriction site for the XhoI enzyme, in position 8887 of the viral genome;
m. Renilla is the gene that codes the luciferase reporter protein Renilla, and that has been cloned in restriction sites NotI-XhoI in position 5′ and 3′, respectively;
n. LTR, whose 5′ end overlaps with the 3′ end of nef element; and
o. LacZ gene cloned in NcoI-AgeI restriction sites in positions 5′ and 3′, respectively, wherein c.1) applies, said LacZ gene is cloned between positions 2593 and 3485, substituting the fragment of pol gene that encode the reverse transcriptase region of the viral genome. This option is described as o.3 when defined previously in the general structure of the HIV-based recombinant viral clone.

### IP HIV NL LacZ/gag-pr Ren (CECT 5848)

Recombinant viral clone based on the general structure previously described, wherein applies b.1), c.4), c.5) and o.2) described previously when defining the general structure of the HIV-based recombinant viral clone, characterized in that it possesses unique restriction sites, introduced by directed mutagenesis, for NarI and KspI enzymes in positions 637 and 4498 of the DNA sequence, respectively, and the LacZ gene cloned between restriction sites ApaI-NcoI in positions 5′ and 3′, respectively, substituting the fragment of pol gene that encodes the protease (FIG. 13).

Within the meaning of the present invention, IP HIV NL LacZ/gag-pr Ren (CECT 5848) refers to a recombinant HIV-based viral clone comprising the following elements in 5′ to 3′ direction:

a. LTR or redundant terminal sequences (R) of the proviral DNA of NL4.3;
b. gag gene which codes the p55 capsid protein formed by the 3 protein subunits MA, CA and NC, and comprising NarI restriction site at position 637 made unique by directed mutagenesis. This option is described as b.1 when defined previously in the general structure of the HIV-based recombinant viral clone;
c. pol gene which codes the viral enzymes needed for the viral replication process, and whose 5′ end overlaps with gag element, and which comprises KspI restriction site generated by directed mutagenesis at position 4498, and ApaI-NcoI restriction sites generated by directed mutagenesis at positions 2006 and 2593 respectively. These options are described as c.4 and c.5 when defined previously in the general structure of the HIV-based recombinant viral clone;
d. vif gene that codes the protein Vif, it's 5′ end overlaps with pol element and it's 3′ end overlaps vpr element;
e. vpr gene that codes the protein Vpr and it's 5′ end overlaps vif element;
f. tat is the gene that codes the protein Tat, it's second exon is contained inside env sequence;
g. vpu is the gene that codes Vpu;
h. env is the gene which codes the protein gp160 of the viral envelope;
i. rev is the gene that codes the protein Rev, it's second exon is contained inside env sequence;
j. nef is the gene that codes protein Nef, and is truncated at the bases in positions 8796 and 8887 of the viral genome;
k. NotI is a restriction site for NotI enzyme, that has been introduced by directed mutagenesis at position 8796 of the viral genome;
l. XhoI is a restriction site for the XhoI enzyme, in position 8887 of the viral genome;
m. Renilla is the gene that codes the luciferase reporter protein Renilla, and that has been cloned in restriction sites NotI-XhoI in position 5′ and 3′, respectively;
n. LTR, whose 5′ end overlaps with the 3′ end of nef element; and
o. LacZ gene cloned in ApaI-NcoI restriction sites in positions 5′ and 3′, respectively, wherein said LacZ gene is cloned between positions 2006 and 2593, substituting the fragment of pol gene that encode the protease region of the viral genome. This option is described as o.2 when defined previously in the general structure of the HIV-based recombinant viral clone.
This option is described as o.1 when defined previously in the general structure of the HIV-based recombinant viral clone.

### IP HIV NL LacZ/env Ren (CECT 5844)

Recombinant viral clone based on the general structure previously described, characterized in that it possesses a unique restriction site for the XbaI enzyme introduced by directed mutagenesis in position 6112 of the DNA sequence, so as to allow the cloning of the envelope gene from the patient's virus, and also the LacZ gene cloned between restriction sites XbaI-NotI in positions 5′ and 3′, respectively, substituting env gene (FIG. 14).

Within the meaning of the present invention, IP HIV NL LacZ/env Ren (CECT 5844) refers to a recombinant HIV-based viral clone comprising the following elements in 5′ to 3′ direction:

a. LTR or redundant terminal sequences (R) of the proviral DNA of NL4.3;
b. gag is the gene which codes the p55 capsid protein formed by 3 protein subunits (MA, CA and NC);
c. pol is the gene which codes the viral enzymes needed for the viral replication process: protease (PRO), reverse transcriptase (RT) and integrase, and whose 5′ end overlaps with gag element;
d. vif is the gene that codes the protein Vif, it's 5′ end overlaps with pol element and it's 3′ end overlaps vpr element;
e. vpr is the gene that codes the protein Vpr and it's 5′ end overlaps vif element;
f. tat is the gene that codes the protein Tat, it's second exon is contained inside env sequence;
g. vpu is the gene that codes Vpu;
h. env is the gene which codes the protein gp160 of the viral envelope, comprising XbaI-NotI restriction sites generated by directed mutagenesis at positions 6114 and 8796 respectively. This option is described as h.1 when defined previously in the general structure of the HIV-based recombinant viral clone;
i. rev is the gene that codes the protein Rev, it's second exon is contained inside env sequence;
j. nef is the gene that codes protein Nef, and is truncated at the bases in positions 8796 and 8887 of the viral genome;
k. NotI is a restriction site for NotI enzyme, that has been introduced by directed mutagenesis at position 8796 of the viral genome;
l. XhoI is a restriction site for the XhoI enzyme, in position 8887 of the viral genome;
m. Renilla is the gene that codes the luciferase reporter protein Renilla, and that has been cloned in restriction sites NotI-XhoI in position 5′ and 3′, respectively;
n. LTR, whose 5′ end overlaps with the 3′ end of nef element; and
o. LacZ gene cloned in XbaI-NotI restriction sites in positions 5′ and 3′, respectively, wherein h.1) applies, said LacZ gene is cloned between positions 6114 and 8796, substituting the fragment of env gene of the viral genome. This option is described as o.4 when defined previously in the general structure of the HIV-based recombinant viral clone.

### IP HIV JRRen (CECT 5843)

Recombinant viral clone based on the general structure previously described, characterized in that it possesses a unique restriction site for the XbaI enzyme introduced by directed mutagenesis in position 6112 of the DNA sequence; the LacZ gene cloned substituting env gene; and the gene “env JR-CSF”, env gene from the clone JR-CSF, substituting the original env gene. This clone is represented in FIG. 15.

The specific position of the restriction sites generated by directed mutagenesis (defined in o.1) to o.4) described previously when defining the general structure of the HIV-based recombinant viral clone) indicates the alternative positions of clonation of the LacZ gene in the claimed HIV-based recombinant viral clone, substituting fragments of gag, pol, and env genes of the viral genome in order to allow further replacement of such sequences with fragments from isolates coming from patients. Therefore, all the elements named therein: 5′ and 3′ LTR, vif, vpr, tat, vpu, rev and nef genes, NotI and XhoI restrictions sites, the Renilla gene and gag, pol, env and LacZ gen, are mandatory shared by all the HIV-based recombinant viral clones subject matter of the present invention and named: CECT 5844, CECT 5845, CECT 5846, CECT 5847, and CECT 5848. The following table (Table I) shows the alternative positions of the specific restriction sites in each recombinant viral clone subject matter of the present invention:

TABLE I

| | | | | | Sequence |
|---|---|---|---|---|---|
| | Element | Element | Element | Element | fragments for |
| Viral | b) gag | c) pol | h) env | o) LacZ | further |
| clone | gene | gene | gene | gene | replacement |
| CECT | | c.1) | | o.3) | Fragment of pol |
| 5845 | | NcoI- | | NcoI- | gene that |
| | | AgeI | | AgeI | encodes the |
| | | | | | Reverse |
| | | | | | Transcriptase |
| | | | | | between |
| | | | | | positions 2593-3485 |
| CECT | | c.2) | | o.2) | Fragment of pol |
| 5846 | | ApaI- | | ApaI- | gene that |
| | | NcoI | | NcoI | encodes the |
| | | | | | Protease between |
| | | | | | positions 2006-2593 |
| CECT | | c.3) | | o.1) | Fragment of pol |
| 5847 | | ApaI- | | ApaI- | gene that |
| | | AgeI | | AgeI | encodes the |
| | | | | | Protease and the |
| | | | | | Reverse |
| | | | | | Transcriptase |
| | | | | | between |
| | | | | | positions 2006-3485 |
| CECT | b.1) | c.4)- | | o.2) | Fragment of pol |
| 5848 | NarI | KspI | | ApaI- | gene that |
| | | c.5) | | NcoI | encodes the |
| | | ApaI- | | | Protease between |
| | | NcoI | | | positions 2006-2593 |
| CECT | | | h.1) | o.4) | Fragment of env |
| 5844 | | | XbaI- | XbaI- | gene that |
| | | | NotI | NotI | encodes the Env |
| | | | | | protein between |
| | | | | | positions 6114-8796 |

The recombinant viral clones of the present invention have shown themselves very useful in the development or improvement of analytical methods and techniques related to investigations surrounding AIDS. In fact, in the specific techniques that were described in the section on State of the Art, said clones have meant major advantages, some of which are detailed below:

Systems for Determination of Phenotypic Resistance to Antiretroviral Drugs:

Phenotypic resistance to antiretroviral drugs is defined by the capacity of a natural or recombinant virus to grow in the presence of a given drug or compound. It is expressed by the “IC50 parameter” (Inhibitory concentration 50) which reflects the concentration of the drug that inhibits 50% of viral replication as compared with replication of the same virus in the absence of any drug.

The present invention refers to a method of using a recombinant viral clone selected from the group consisting of: clone IP HIV NL LacZ/rt Ren (CECT 5845), clone IP HIV NL LacZ/pr Ren (CECT 5846), clone IP HIV NL LacZ/pol Ren (CECT 5847), clone IP HIV NL LacZ/gag-pr Ren (CECT 5848), and clone IP HIV NL LacZ/env Ren (CECT 5844), to determine phenotypic resistances to antiretroviral drugs for the treatment of HIV infection, comprising the following steps:

a) extraction of RNA from the HIV from the patient's plasma,
b) retrotranscription and amplification of said patient's viral RNA using specific primers for each viral gene by means of nested polymerase chain reaction, said primers including specific restriction sites generated by directed mutagenesis, said specific restriction sites being select from the following group:
b.i) NarI-KspI at positions 637 and 4498 respectively and ApaI-NcoI restriction sites at positions 2006 and 2593 respectively, for later cloning the amplificate patient's fragment of gag-pol gene between such positions replacing the LacZ gene of the recombinant viral clone IP HIV NL LacZ/gag-pr Ren (CECT 5848),
b.ii) NcoI-AgeI restriction sites at positions 2593 and 3485 respectively, for later cloning the amplificate patient's fragment of pol gene between such positions replacing the LacZ gene of the recombinant viral clone IP HIV NL LacZ/rt Ren (CECT 5845),
b.iii) ApaI-NcoI restriction sites at positions 2006 and 2593 respectively, for later cloning the amplificate patient's fragment of pol gene between such positions replacing the LacZ gene of the recombinant viral clone IP HIV NL LacZ/pr Ren (CECT 5846),
b.iv) ApaI-AgeI restriction sites at positions 2006 and 3485 respectively, for later cloning the amplificate patient's fragment of pol gene between such positions replacing the LacZ gene of the recombinant viral clone IP HIV NL LacZ/pol Ren (CECT 5847, and
b.v) XbaI-NotI restriction sites at positions 6114 and 8796 respectively, for later cloning the amplificate patient's fragment of env gene between such positions replacing the LacZ gene of the recombinant viral clone IP HIV NL LacZ/env Ren (CECT 5844),
   c) enzymatic digestion of the amplificate and the recombinant viral clone and in vitro ligation process carried out using the T4 ligase for generating the recombinant viral clone carrying corresponding amplified fragment from the patient as defined in b.i) to b.v),
   d) the generated recombinant viral clone described in c) is transfected in the 293T cell line,
   e) the infectious progeny of the generated recombinant viral clone described in d) is gathered 48 hours after the transfection and is used for infecting the SSPA-B7 cell line,
   f) determining the resistance to Protease inhibitors, Reverse Transcriptase inhibitors by evaluating the IC50 in the infection of the SSPA-B7 target cells in comparison with a recombinant viral clone selected from the group consisting of: clone IP HIV NL LacZ/rt Ren (CECT 5845), clone IP HIV NL LacZ/pr Ren (CECT 5846), clone IP HIV NL LacZ/pol Ren (CECT 5847), clone IP HIV NL LacZ/gag-pr Ren (CECT 5848), and clone IP HIV NL LacZ/env Ren (CECT 5844), without any associated resistance mutations, and
   g) reading the sensitivity to said Protease inhibitors, Reverse Transcriptase inhibitors, by quantifying the capacity of the generated recombinant viral clone described in e) to complete a replication cycle which is quantified by measuring the renilla activity in the infected SSPA-B7 target cells by means of a luminometer.

The proposed invention is based on the system of cloning HIV gene fragments of reverse transcriptase, of the envelope and of Protease into viral vectors that contain marker genes. This invention presents a series of advantages with respect to those already existing, namely:
a) The possibility of separately analysing the resistance to inhibitors of Protease, of Reverse Transcriptase and of the envelope. This makes it possible to perform an independent evaluation of resistances to different pharmacological groups.
b) the use of multiple cycle viral systems.
c) A greater efficacy in the evaluation of viral isolates with low replicative capacity.

Systems for Determination of HIV Replicative Capacity:

Replicative capacity of HIV or viral fitness is the final result of a multiple set of characteristics of the virus in the process of adaptation to its host. It reflects the capacity of replication of a given viral strain or recombinant virus under a particular host environment. It also refers to the capacity of replication (higher or lower) of a given virus in comparison with another viral strain. In this case we said that viral strain 1 is more/less fit than viral strain 2.

The present invention refers to a method of using a recombinant viral clone selected from the group consisting of: clone IP HIV NL LacZ/rt Ren (CECT 5845), clone IP HIV NL LacZ/pr Ren (CECT 5846), clone IP HIV NL LacZ/pol Ren (CECT 5847), clone IP HIV NL LacZ/gag-pr Ren (CECT 5848), and clone IP HIV NL LacZ/env Ren (CECT 5844), for determining the replicative capacity in a generated recombinant viral clone carrying a sequence fragment from a patient with HIV infection, said method comprising the following steps:

a) extraction of RNA from the HIV from the patient's plasma,

b) retrotranscription and amplification of said patient's viral RNA using specific primers for each viral gene by means of nested polymerase chain reaction, said primers including specific restriction sites generated by direct mutagenesis, said specific restriction sites being select from the following group:
b.i) NarI-KspI at positions 637 and 4498 respectively and ApaI-NcoI restriction sites at positions 2006 and 2593 respectively, for later cloning the amplificate patient's fragment of gag-pol gene between such positions replacing the LacZ gene of the recombinant viral clone IP HIV NL LacZ/gag-pr Ren (CECT 5848),
b.ii) NcoI-AgeI restriction sites at positions 2593 and 3485 respectively, for later cloning the amplificate patient's fragment of pol gene between such positions replacing the LacZ gene of the recombinant viral clone IP HIV NL LacZ/rt Ren (CECT 5845),
b.iii) ApaI-NcoI restriction sites at positions 2006 and 2593 respectively, for later cloning the amplificate patient's fragment of pol gene between such positions replacing the LacZ gene of the recombinant viral clone IP HIV NL LacZ/pr Ren (CECT 5846),
b.iv) ApaI-AgeI restriction sites at positions 2006 and 3485 respectively, for later cloning the amplificate patient's fragment of pol gene between such positions replacing the LacZ gene of the recombinant viral clone IP HIV NL LacZ/pol Ren (CECT 5847), and
b.v) XbaI-NotI restriction sites at positions 6114 and 8796 respectively, for later cloning the amplificate patient's fragment of env gene between such positions replacing the LacZ gene of the recombinant viral clone IP HIV NL LacZ/env Ren (CECT 5844),
   c) enzymatic digestion of the amplificate and the recombinant viral clone, and in vitro ligation process carried out using the T4 ligase for generating the recombinant viral clone carrying corresponding amplified fragment from the patient as defined in b.i) to b.v),
   d) the generated recombinant viral clone described in c) is transfected in the 293T cell line,
   e) the infectious progeny of the generated recombinant viral clone described in d) is gathered 48 hours after the transfection and is used for infecting the SSPA-B7 cell line,
   f) infecting said SSPA-B7 cell cultures with the cited generated recombinant viral clone described in e) or with the wild virus, and
   g) measuring luciferase activity produced in the infected SSPA-B7 target infected cells described in step
   f) by quantifying the capacity of the generated recombinant viral clone described in step f) to complete a replication cycle which is quantified by measuring the renilla activity by means of a luminometer.

The proposed invention permits this parameter to be determined and a direct analysis to be made of the viral replicative capacity in target cells very close to physiological targets such as peripheral blood lymphocytes. The cloning of the envelope genes and different fragments of the patient's gag-pol DNA in multiple cycle carrier viruses of marker genes (Renilla) confers the chimera virus with the replicative properties of the mutated virus. Generation of multiple cycle viruses is particularly important in determining viral fitness because a decreased in viral fitness is associated by different viral mutations, particularly those related to resistance to antiretroviral drugs. This “slow-replicating” or “low-fit” viruses grow with difficulty in culture and their detection can be missed if single cycles of replications are used. In contrast, multiple cycles of infection and replication allows progression of the low-fit viruses in culture and their detection by sensitive techniques as luciferase detection. Unlike the evaluation systems for viral fitness, which are extremely laborious, the development permits analysis of the replicative capacity of the recombinant virus in a manner that is virtually continuous.

Systems for Determination of the Presence of Neutralising Antibodies:

Neutralising antibodies are antibodies that have the capacity of binding to the viral envelope and that inhibit the infection of target cells.

The present invention refers to a method of using the recombinant viral clone IP HIV NL LacZ/env Ren (CECT 5844), to detect neutralising antibodies against HIV, comprising the following steps:

a) obtaining serum of seropositive patients for HIV and of non-infected individuals subjected to vaccination,
b) extraction of RNA from the HIV from the patient'serum,
c) retrotranscription and amplification of said patient's viral RNA using specific primers by means of nested polymerase chain reaction, said primers including XbaI-NotI restriction sites generated by direct mutagenesis at positions 6114 and 8796 respectively, for later cloning the amplified fragment of env gene between such positions replacing the LacZ gene of the recombinant viral clone,
d) enzymatic digestion of the amplificate and the recombinant viral clone, and in vitro ligation process carried out using the T4 ligase for generating the recombinant viral clone carrying corresponding amplified fragment from the patient as defined in c),
e) the generated recombinant viral clone described in d) is transfected in the 293T cell line,
f) the infectious progeny of the generated recombinant viral clone defined in e) is gathered 48 hours after the transfection and is used for infecting the SSPA-B7 cell line carrying either the CCR5 or the CXCR4 receptor for HIV-1, and
g) evaluation of the neutralising capacity of said patients serum towards the generated recombinant viral clone defined in f) by direct analyzing the viral replication and its inhibition by serial sera dilutions from said patients by measuring the luciferase activity produced in the infected SSPA-B7 target infected cells described in step f) by quantifying the capacity of the generated recombinant viral clone described in step f) to complete a replication cycle which is quantified by measuring the renilla activity by means of a luminometer.

The proposed invention permits the two main drawbacks of classical techniques for determining neutralising capacity in the serum of seropositive patients to be overcome, since it enables a direct analysis to be made of viral replication and its inhibition by the patient's antibodies. It is possible to do this both on isolates or reference viral clones, as well as on a recombinant virus in which the envelope of the viral clone has been substituted by the complete envelope of the patient's viral population.

This type of assay, known by the applicant as “autologous test for detection of neutralising antibodies”, has a high sensitivity and allows a precise evaluation of the neutralising capacity of the patient's serum towards the viruses that are replicating in his organism at the moment of the test. Within the meaning of this invention, neutralisation tests or tests for detection of neutralising antibodies refers to tests for detecting the presence of antibodies either in naturally-infected patients or induced by vaccines that are able to block the entry of the virus into the cell. Cells and virus are cultured in vitro in the presence of sera and the inhibition of viral infection by serial sera dilutions is quantified. Results as summarized as IC50 that reflects the dilution of sera that neutralizes the viral infection by 50% in comparison with virus cultured in the absence of serum.

Systems for the Characterisation of Viral Tropism in Infection by HIV:

Viral tropism refers to the capacity of HIV to enter into the cell through two different receptors named CCR5 and CXCR4. According to their tropism, viral strains are classified according to their capacity to infect the cell through the CCR5 receptor (R5 strains), the CXCR4 receptor (X4 strains) or both receptors (named dual-tropic or R5X4 strains).

The present invention refers to a method of using the recombinant viral clone IP HIV NL LacZ/env Ren (CECT 5844), to characterise viral tropism in HIV infection, comprising the following steps:

a) extraction of RNA from the HIV from the patient's plasma,
b) retrotranscription and amplification of said patient's viral RNA using specific primers by means of nested polymerase chain reaction, said primers including XbaI-NotI restriction sites generated by direct mutagenesis at positions 6114 and 8796 respectively, for later cloning the amplified fragment of env gene between such positions replacing the LacZ gene of the recombinant viral clone,
c) enzymatic digestion of the amplificate and the recombinant viral clone, and in vitro ligation process carried out using the T4 ligase for generating the recombinant virus clone carrying corresponding amplified fragment from the patient as defined in b),
d) the generated recombinant virus clone described in c) is transfected in the 293T cell line,
e) the infectious progeny of the generated recombinant viral clone defined in d) is gathered 48 hours after the transfection and is used for infecting target cells carrying either the CCR5 or the CXCR4 receptor for HIV-1, and
f) characterizing the viral tropism of the generated recombinant viral clone defined in e) by measuring the luciferase activity produced in said target cells, by quantifying the capacity of said generated recombinant viral clone to complete a replication cycle which is quantified by measuring the renilla activity by means of a luminometer.

The proposed invention permits this parameter to be determined by means of two tools: the generation of recombinant viruses which carry the complete envelope of the patient's viral population and the use of target cells which stably expresses either the CCR5 or the CXCR4 receptor.

Experimental Models for the Screening of Compounds with Potential Antiviral Activity:

The antiviral activity of a given compound that can be a plant extract, a chemical molecule or a bio-engineered product can be assessed in vitro culturing the virus and target cells in the presence of growing concentrations of the compound. Antiviral activity is summarized as IC50 value, which represents the concentration of the drug that is able to inhibit by 50% the infection as compared with virus cultured in the absence of the compound.

The present invention refers to a method of using a recombinant viral clone selected from the group consisting of: clone IP HIV NL LacZ/rt Ren (CECT 5845), clone IP HIV NL LacZ/pr Ren (CECT 5846), clone IP HIV NL LacZ/pol Ren (CECT 5847), clone IP HIV NL LacZ/gag-pr Ren (CECT 5848), and clone IP HIV NL LacZ/env Ren (CECT 5844), to screen and characterise compounds for antiviral activity towards HIV, comprising the following steps:

a) extraction of RNA from the HIV from the patient's plasma,
b) retrotranscription and amplification of said patient's viral RNA using specific primers for each viral gene by means of nested polymerase chain reaction, said primers including specific restriction sites generated by direct mutagenesis, said specific restriction sites being select from the following group:
b.i) NarI-KspI at positions 637 and 4498 respectively and ApaI-NcoI restriction sites at positions 2006 and 2593 respectively, for later cloning the amplificate patient's fragment of gag-pol gene between such positions replacing the LacZ gene of the recombinant viral clone IP HIV NL LacZ/gag-pr Ren (CECT 5848),
b.ii) NcoI-AgeI restriction sites at positions 2593 and 3485 respectively, for later cloning the amplificate patient's fragment of pol gene between such positions replacing the LacZ gene of the recombinant viral clone IP HIV NL LacZ/rt Ren (CECT 5845),
b.iii) ApaI-NcoI restriction sites at positions 2006 and 2593 respectively, for later cloning the amplificate patient's fragment of pol gene between such positions replacing the LacZ gene of the recombinant viral clone IP HIV NL LacZ/pr Ren (CECT 5846),
b.iv) ApaI-AgeI restriction sites at positions

2006 and 3485 respectively, for later cloning the amplificate patient's fragment of pol gene between such positions replacing the LacZ gene of the recombinant viral clone IP HIV NL LacZ/pol Ren (CECT 5847), and
b.v) XbaI-NotI restriction sites at positions

6114 and 8796 respectively, for later cloning the amplificate patient's fragment of env gene between such positions replacing the LacZ gene of the recombinant viral clone IP HIV NL LacZ/env Ren (CECT 5844),

c) enzymatic digestion of the amplificate and the recombinant viral clone, and in vitro ligation process carried out using the T4 ligase for generating the recombinant viral clone carrying corresponding amplified fragment from the patient as defined in b.i) to b.v),
d) the generated recombinant viral clone described in c) is transfected in 293T cells,
e) the infectious progeny of the generated recombinant viral clone described in d) is gathered 48 hours after the transfection and is used for infecting the SSPA-B7 cell line, in the presence of compounds with potential antiviral activity,
f. screening and characterising compounds with potential antiviral activity by analyzing the direct inhibition of HIV replication by different concentrations of a given compound through measuring the luciferase activity produced in the SSPA-B7 target infected cells defined in e) by quantifying the capacity of the generated recombinant viral clone to complete a replication cycle which is quantified by measuring the renilla activity by means of a luminometer.

The proposed invention permits the detection of antibody activity to be carried out in an easily robotisable microplate format, in a model which covers the entire viral replicative cycle by means of using multiple cycle vectors. Current systems detecting antiviral activity are based on the protection conferred by a given compound from the cytopathic effect induced by HIV replication in cell culture. Thus, antiviral activity is measured through an indirect parameter (protection from cell killing) that requires at least a seven days culture for its assessement. In contrast the proposed system permits an analysis of the direct inhibition of HIV replication through the measurement of luciferase activity in cell lysates that reflects in a highly sensitive manner (100 times more sensitive than measurement of cytopathic effect) the degree of HIV replication and its inhibition by a given compound. Besides, this method considerably cuts down screening times to 24 or 48 hours.

Thus, the present invention also relates to the use of the previously described viral clones, in analytical methods for the determination of phenotypic resistances to antiretroviral compounds for treatment of HIV infection.

A specific realization of the invention refers to the use of said viral clones in analytical methods for the determination of the replicative capacity of the recombinant virus that carry the gag, pol and/or env sequences from patients with HIV infection.

On the other hand, the present invention relates to the use of said recombinant viral clones in analytical methods for the characterization of viral tropism in HIV infection.

In another particular embodiment, the present invention realtes to the use of said recombinant viral clones in analytical methods for the detection of neutralizing antibodies against HIV in the serum of patients seropositive for HIV and non-infected individuals, subjected to vaccination or otherwise.

Lastly, the present invention refers to the use of said recombinant viral clones in analytical methods for the screening and characterization of compounds with antiviral activity against HIV.

### BRIEF DESCRIPTION OF THE FIGURES

FIGS. 1a and 1b: Illustrative diagrams corresponding to the production of viral clones of the present invention, in accordance with the process described in preferred embodiment 1. FIG. 1a shows a diagram of the proccess for obtaining the chimeric clones from the patient's serum and the recombinant viral clones. FIG. 1b explains how it is produced the transfection and the infection of the target cells from an initial chimeric viral clone with the proteasa and the reverse transcriptase fragments from the patient, in order to measure the renilla activity in the presence of protease and reverse transcriptase inhibitors.

FIGS. 2a and 2b: Graphic representations corresponding to the results of studies discussed in section 2.1 of Modes of Embodiment of the Invention. FIG. 2a represents the phenotypic profile of sensitivity of the viral clone IP HIV NL Ren towards the following drugs: inhibitors of reverse transcriptase analogous to 3TC nucleosides (A), AZT/ZDV (B), d4T (C), ddI (D), inhibitors of reverse transcriptase not analogous to nucleosides; Efavirenz (E); inhibitors of protease: Saquinavir (F). FIG. 2b is a graphic representation illustrating a study of the determination of AZT resistance in a wild type virus (solid line) and in a virus with the mutations M41L, K07R, T215F, K219Q (broken line): Fold=36

FIG. 3: Graphic representation corresponding to the determination studies of replicative capacity discussed in section 2.2 of Modes of Embodiment of the Invention. Histogram showing the improvement in the recovery of a virus with multiple resistance mutations in the Protease and RT when separate cloning is carried out of both fragments than with the complete pol gene.

FIG. 4: Expression of CCR5 and CXCR4 by the SSPA-B7 clone in accordance with section 2.3 and 2.4 of Modes of Embodiment of the Invention. A cellular clone of SSPA-B7 has been generated by means of genetic engineering techniques which expresses the receptor CCR5.

FIG. 5: Cytopathic effect induced in the clone SSPA-B7 by the isolates NL4.3 (X4) and Bal (R5), in accordance with sections 2.3 and 2.4 of Modes of Embodiment of the Invention. A cellular clone of SSPA-B7 has been generated by means of genetic engineering techniques which expresses the receptor CCR5 and which is susceptible to infection by the virus R5, X4 or R5X4.

FIG. 6: Analysis of the neutralising capacity of the NL-Luc virus of a patient's plasma under the conditions of section 2.4(D) of Modes of Embodiment of the Invention, showing the results of the tests conducted with the viral clones according to the inventive system for the detection and titration of neutralising antibodies of the present invention.

FIG. 7: Results of the analysis of antiviral activity of two compounds studied according to section 2.5(C) of Modes of Embodiment of the Invention showing graphic representations corresponding to the analysis of antiviral activity of two compounds derived from plant products.

FIG. 8: General structure of recombinant viral clones of the present invention.

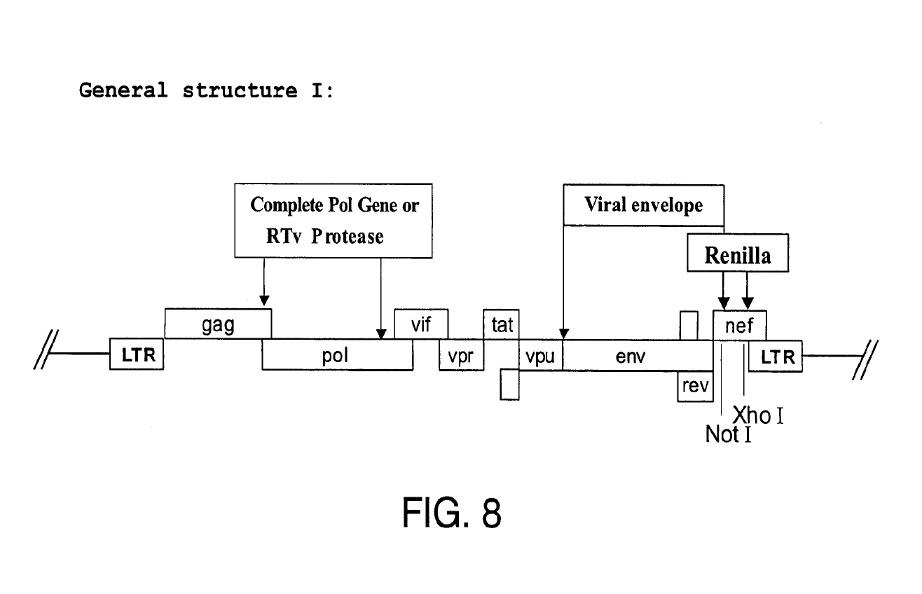

FIG. 9: Recombinant viral clone IP HIV NL Ren, deposited in the Spanish Collection of Type Cultures as CECT 5842, where ApaI and AgeI represent unique restriction sites in the DNA sequence and the remaining symbols have the meaning given above for FIG. 8.

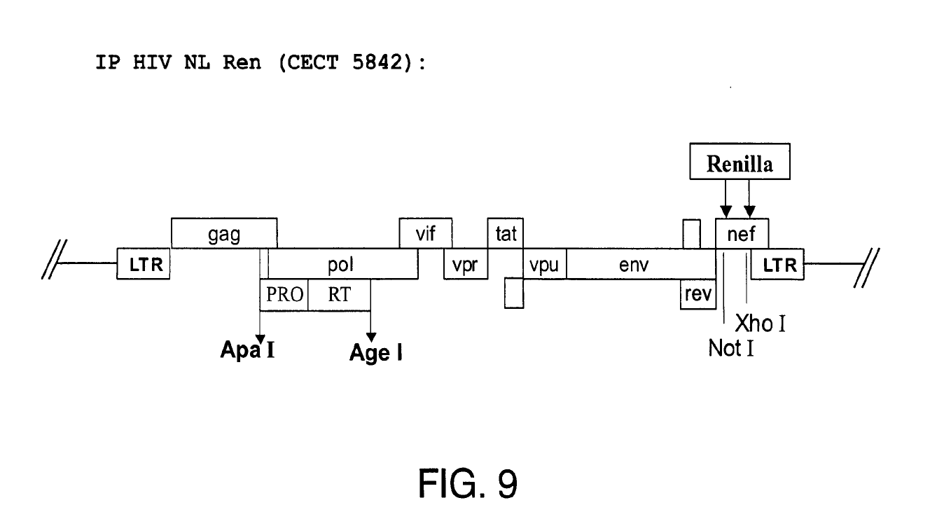

FIG. 10: Recombinant viral clone IP HIV NL LacZ/pol Ren, deposited in the Spanish Collection of Type Cultures as CECT 5847, where LacZ indicates the cloning position of the gene LacZ substituting a fragment of the pol gene, and the remaining symbols have the meaning given above.

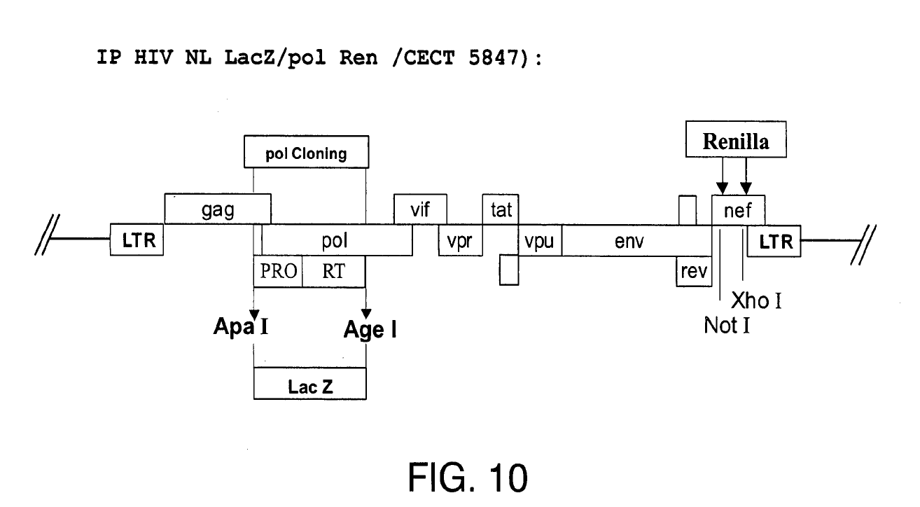

FIG. 11: Recombinant viral clone IP HIV NL LacZ/pr Ren, deposited in the Spanish Collection of Type Cultures as CECT 5846, where NcoI indicates a unique restriction site in the DNA sequence, and the remaining symbols have the meaning given above.

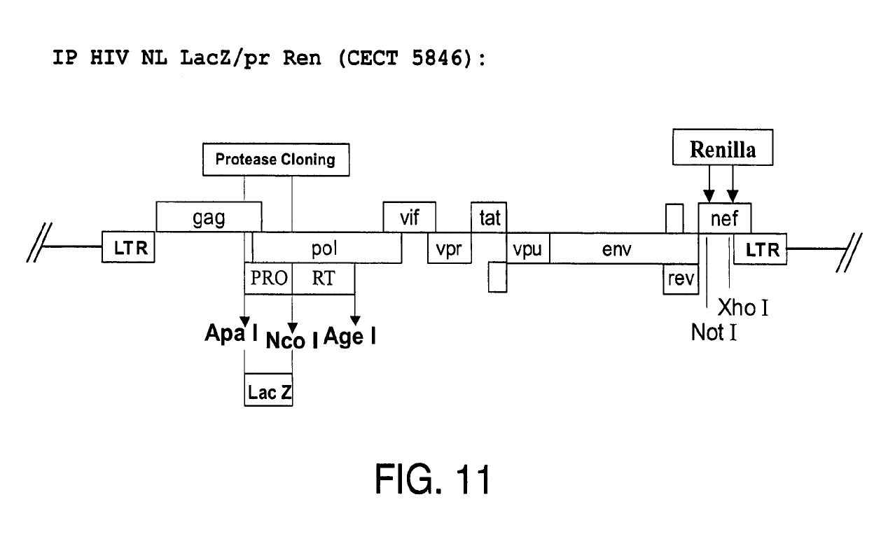

FIG. 12: Recombinant viral clone IP HIV NL LacZ/rt Ren, deposited in the Spanish Collection of Type Cultures as CECT 5845, where the different symbols have the same meaning as above.

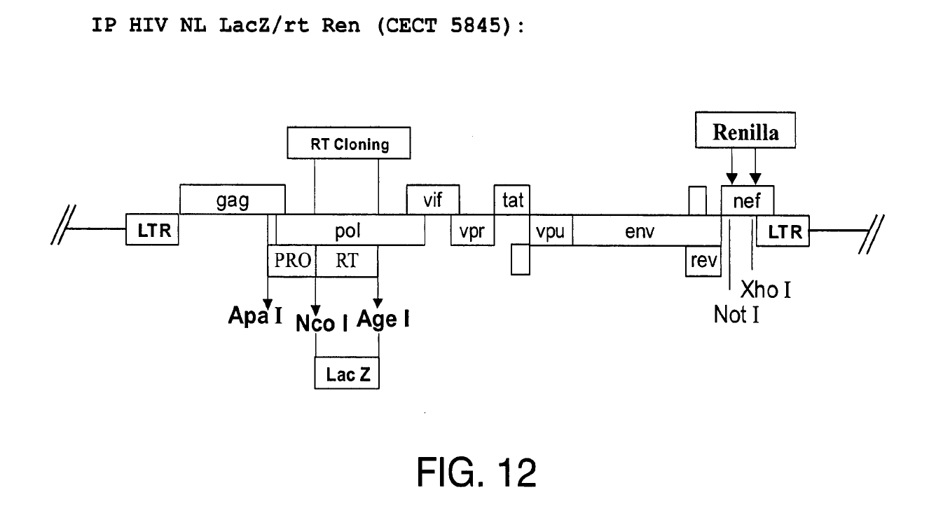

FIG. 13: Recombinant viral clone IP HIV NL LacZ/gag-pr Ren, deposited in the Spanish Collection of Type Cultures as CECT 5848, where NarI and KspI indicate unique restriction sites in the DNA sequence, and the remaining symbols have the meaning given above.

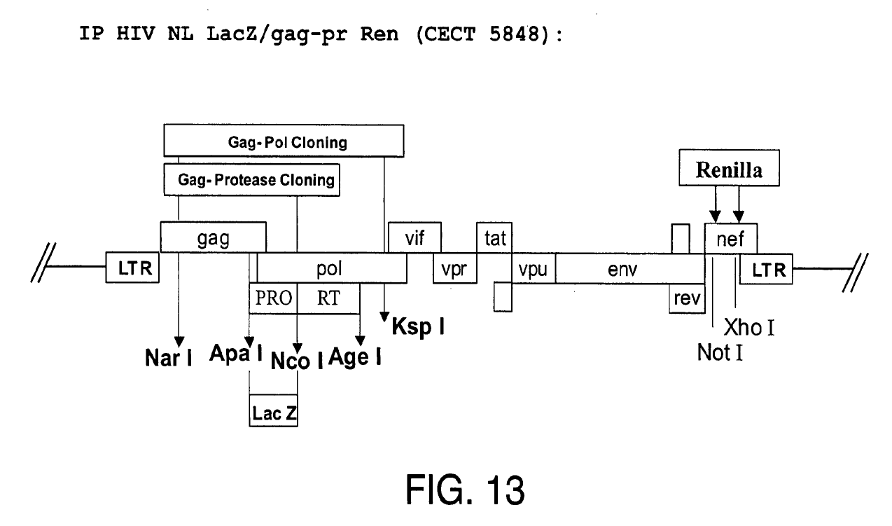

FIG. 14: Recombinant viral clone IP HIV NL LacZ/env Ren, deposited in the Spanish Collection of Type Cultures as CECT 5844, where XbaI indicates a unique restriction site in the DNA sequence, “patient env” indicates the cloning position of the patient's gene, and the remaining symbols have the meaning given above.

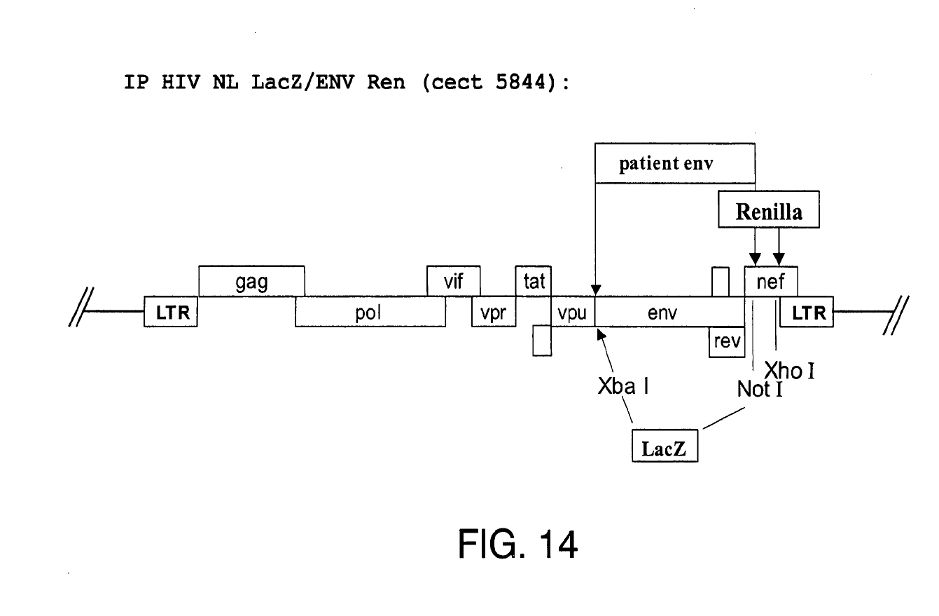

FIG. 15: Recombinant viral clone IP HIV JRRen, deposited in the Spanish Collection of Type Cultures as CECT 5843, where XbaI indicates a unique restriction site in the DNA sequence, “env JR-CSF” indicates the cloning position of the env gene of the clone JR-CSF in place of the envelope of NL 4.3 and the remaining symbols have the meaning given above.

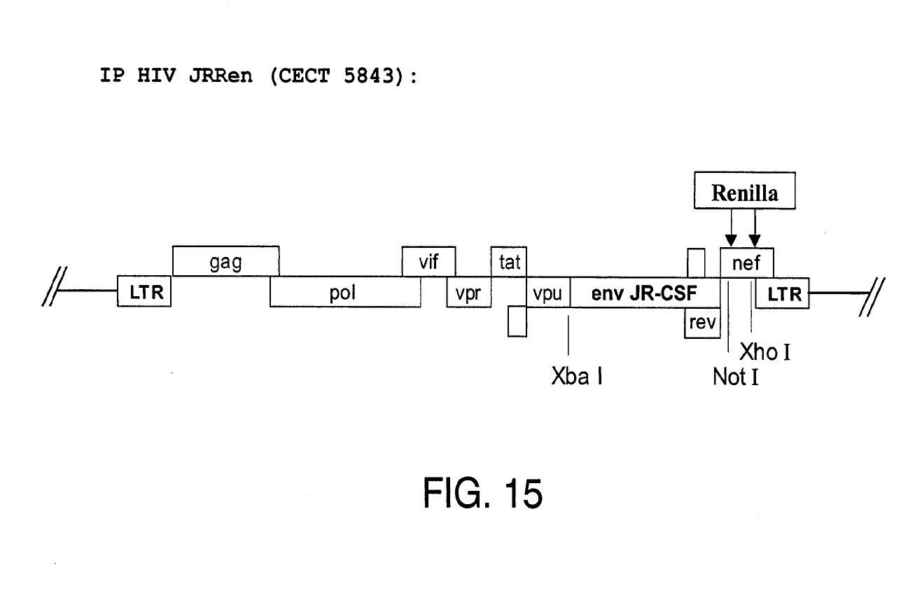

### MODES OF EMBODIMENT OF THE INVENTION

The present invention is illustrated forthwith by means of a detailed description of preferred embodiments, in which the recombinant viral clones of the invention are shown along with the main applications together with some of the general techniques of genetic engineering used in the different cases, all this making use of the attached figures for greater clarity. It is understood that the skilled in the art will be able to understand the modifications, variations and changes that can be implemented within the scope of the present invention.

### EXAMPLES

### Example 1

### Obtaining of Recombinant Viral Clones

This is based on the system of cloning gene fragments corresponding to HIV reverse transcriptase and protease in viral carrier vectors that contain marker genes.

(A) General Description of the Technique:

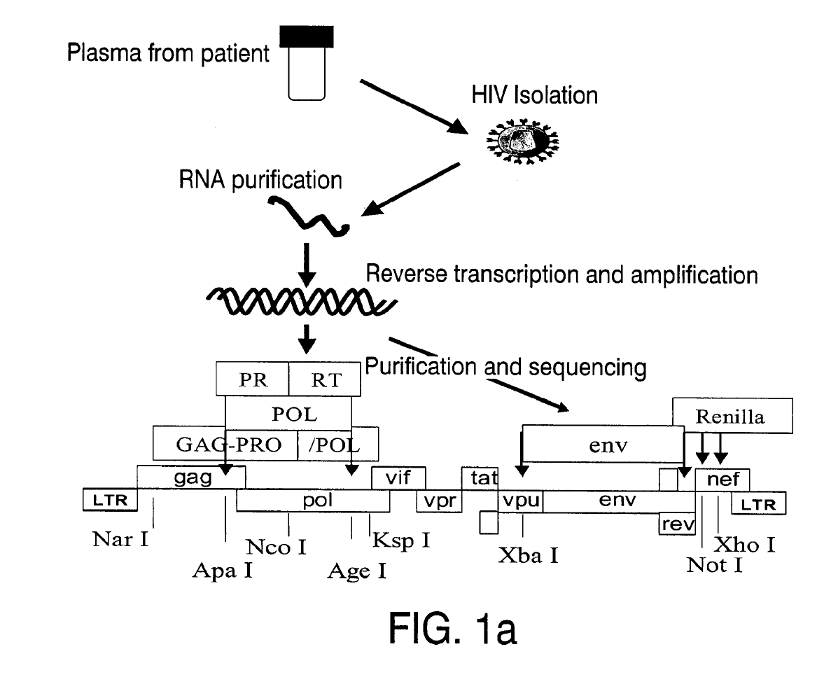

It can be schematically seen in FIGS. 1a and 1b how the viral particles are produced in culture supernatants during the 48 hours following transfection of the recombinat virus carrying viral sequences amplified from patients's plasma in 293T cells. The 293T cell line was obtained from the Deposit of the ATCC. The progeny of recombinant viruses are collected 48 hours after transfection in and used to infect the SSPA-B7 clone as target. This cellular clone was obtained by the applicant from the MT-2 cell line by means of transfection of an expression vector of the gene CCR5 provided with a resistance marker for Genetycin. Expression of both major viral coreceptors (CCR5 as a transgene and CXCR4 naturally) allows the infection with viral carrying different envelopes that use CCR5, CXCR4 receptors or both. The resistance for Geneticin allows the selection of pure populations of double positive cells (CCR5 and CXCR4) through culture in the presence of this antibiotic. The capacity of the viruses to complete a replication cycle is quantified by measuring the luciferase activity in the target cells. When phenotypic resistance to protease inhibitors is analyzed, the drug should be added to the producer cell (293-T) because viral morphogenesis driven by the protease is generated in this step of the viral cycle. In contrast, phenotypic resistance towards inhibitors of reverse transcriptase and of entry is measured by adding the drugs to the infected cells because such processes (entry and retrotranscription) take place in the target cell.

The process comprises the following operations:
Starting from 0.5 ml of plasma from HIV-infected patients, the extraction of RNA from the HIV is carried out.
The viral RNA is retrotranscribed and then amplified using specific primers for each viral gene by means of polymerase chain reaction. The primers include specific restriction sites for subsequent cloning into the different viral constructs generated depending on the type of recombinant virus it is wished to generate.
Following enzymatic digestion of the amplificate and of the reference virus, an in vitro ligation process is carried out using the T4 ligase.
The population of the generated recombinant provirus is transfected in the 293T cell line and acts as a producer cell of recombinant viruses.
The infectious progeny of recombinant viruses is gathered 48 hours after the transfection and is used for infecting the SSPA-B7 cell line.
When the application is the determination of resistance to antiretrovirals, the last two processes are carried out in the presence of protease inhibitors (in the case of 293T producer cells), reverse transcriptase inhibitors (in the case of SSPA-B7 target cells), or viral entry inhibitors (in the case of SSPA-B7 target cells).
The level of resistance to the different drugs is defined by means of the concentration that gives a 50% inhibition of viral replication (IC50) in comparison with a reference virus without any associated resistance mutations.
The reading of the viral growth in the presence of increasing concentrations of the different drugs is done by quantifying the renilla activity. Briefly, 48 hours after infection cells are collected by centrifugation and lysed in buffer containing detergent to inactivate the virus. This cell lisate is incubated with luciferin salt and ATP that are the substrates for the renilla-luciferase enzyme. Luminiscence produced by the reaction, reflects directly the amount of luciferase in culture, which is proportional to the degree of viral replication. Light emission is quantified by means of a Berthold Orion Microplate luminometer.

(B) Virus:
This starts from the proviral vector NL4.3 (Adachi et al. 1986). This clone has been genetically modified in the laboratory by the following procedures: insertion of the renilla-luciferase gene, deletion of specific sequences that are replaced by the lac-Z gene and generation of specific restriction sites through site-directed mutagenesis. The final clones produce multiple cycle viral clones which express the indicator gene Renilla instead of nef and in which different restriction targets have been introduced in order to be able to clone the complete pol gene, the fragments Reverse Transcriptase or Protease separately, the regions gag protease and gag-pol or the complete env gene.

The recombinant viral clones obtained permit cloning of the patient's complete pol gene, the reverse transcriptase and protease separately, the gag region along with the protease or the complete pol gene. It also permits cloning of the patient's complete env gene. All these are multiple cycle viruses and are very useful when multiple resistance mutations in the patient's RT and Protease exist, as the final replicative capacity is improved.

(C) Primers:
In the most important operations mentioned earlier, the following primers and the following cells are used:

Mutagenesis

Mutagenesis Not I:
(SEQ ID NO: 1)
5′ GCTATAAGATGGGTGGCGCGGCCGCAAAAAGTAGTGTGATTGG 3′
(SEQ ID NO: 2)
5′ CCAATCACACTACTTTTTGCGGCCGCGCCACCCATCTTATAGC 3′
Mutagenesis Nco I:
(SEQ ID NO: 3)
5′ CCAGTAAAATTAAAGCCAGCCATGGATGGCCCAAAAG 3′
(SEQ ID NO: 4)
5′ CTTTTGGGCCATCCATGGCTGGCTTTAATTTTACTGG 3′
Mutagenesis Ksp I:
(SEQ ID NO: 5)
5′ GAAGCAGAAGTAATTCCCGCGGAGACAGGGCAAGAAAC 3′
(SEQ ID NO: 6)
5′ GTTTCTTGCCCTGTCTCCGCGGGAATTACTTCTGCTTC 3′
Mutagenesis Nar I:
(SEQ ID NO: 7)
5′ GAAAATACCGCATCAGGACCCATTCGCCATTCAGGC 3′
(SEQ ID NO: 8)
5′ GCCTGAATGGCGAATGGGTCCTGATGCGGTATTTTC 3′
Mutagenesis Xbal:
(SEQ ID NO: 9)
5′ GCATTAGTAGTAGCAATAATAATAGCTCTAGAGCTGTGGTCCATAGTA
ATCATAG
(SEQ ID NO: 10)
5′ CTATGATTACTATGGACCACAGCTCTAGAGCTATTATTATTGCTACTA
CTAATGC

Amplification of the Pol Gene of Patients

POL:
(SEQ ID NO: 11)
5′GCCAAAAATTGCAGGGCCCCTAGG A 3′
(SEQ ID NO: 12)
5′ TCTTTTGATGGGTCATAATACACTCCATGTACCGG 3′
PRO:
(SEQ ID NO: 13)
5′ GCCAAAAATTGCAGGGCCCCTAGGA 3′
(SEQ ID NO: 14)
5′ CATGCCATGGCTGGCTTTAATTTTACTGGTACAGTC 3′
RT:
(SEQ ID NO: 15)
5′ CATGCCATGGATGGCCCAAAAGTTAAACAATGGCC 3′
(SEQ ID NO: 16)
5′ TCTTTTGATGGGTCATAATACACTCCATGTACCGG 3′
GAG-PR:
(SEQ ID NO: 17)
5′ GGAAAATCTCTAGCAGTGGCGCCCGAACAG 3′
(SEQ ID NO: 18)
5′ CATGCCATGGCTGGCTTTAATTTTACTGGTACAGTC 3′
GAG-POL:
(SEQ ID NO: 19)
5′ GGAAAATCTCTAGCAGTGGCGCCCGAACAG 3′
(SEQ ID NO: 20)
5′ CTTGCCCTGTCTCTGCTGGAATTACTTCTGC 3′

Amplification of the Env Gene of Patients

First Amplification:

(SEQ ID NO: 21)
5′ TATGAAACTTACGGGGATACTTGGG 3′
(position 5697-5721 of the pNL4.3)
(SEQ ID NO: 22)
5′ CTGCCAATCAGGGAAGTAGCCTTGTGT 3′
(position 9135-9161 of the pNL4.3)

Nested-PCR:

(XbaI target)
(SEQ ID NO: 23)
5′ GTAGCAATAATAATAGCTCTAGAGCTGTGGTCCATAGTAATC 3′
(position 6097-6138 of pNL4.3)
(Not I target)
(SEQ ID NO: 24)
5′ TACTTTTTGCGGCCGCGCCACCCATCTTATAGC 3′
(position 8779-8811 of the pNL4.3)

(D) HIV-Based Recombinant Viral Clones:

The procedure set out in the above section using primers, probes, target sequences, cell lines and stated conditions has permitted the following viral clones of the present invention to be obtained:

### IP HIV NL Ren: Deposit Number CECT 5842

### IP HIV NL LacZ/pr Ren: Deposit Number CECT 5846

### IP HIV NL LacZ/rt Ren: Deposit Number CECT 5845

### IP HIV NL LacZ/pol Ren: Deposit Number CECT 5847

### IP HIV NL LacZ/gag-pr Ren: Deposit Number CECT 5848

### IP HIV NL LacZ/env Ren: Deposit Number CECT 5844

### IP HIV JRRen: Deposit Number CECT 5843

### Example 2

### Evaluation of the Viral Clones of the Invention in Different Systems of Analytical Determination

### 2.1.—Determination System for Phenotypic Resistances to Antiretroviral Drugs

The tested clones were the following

IP HIV NL Ren,

IP HIV NL LacZ/pol Ren,

IP HIV NL LacZ/pr Ren,

IP HIV NL LacZ/rt Ren,

IP HIV NL LacZ/gag-pr Ren and

IP HIV NL LacZ/env Ren

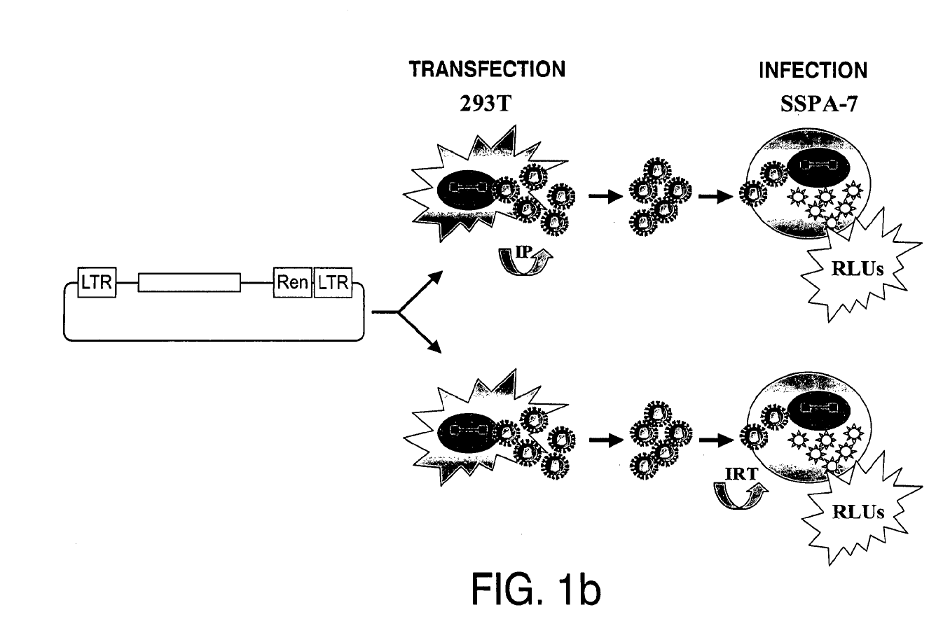

The results of the tests carried out with these viral clones according to the inventive system for the determination of phenotypic resistances to antiretroviral drugs are shown in FIGS. 2a and 2b.

FIG. 2a represents the phenotypic profile of sensitivity of the viral clone IP HIV NL Ren towards the following drugs: inhibitors of reverse transcriptase analogous to 3TC nucleosides (A), AZT/ZDV (B), d4T (C), ddI (D), inhibitors of reverse transcriptase not analogous to nucleosides; Efavirenz (E); inhibitors of protease: Saquinavir (F).

FIG. 2b is a graphic representation illustrating a study of the determination of AZT resistance in a wild type virus (solid line) and in a virus with the mutations M41L, K07R, T215F, K219Q (broken line): Fold=36.

Among the advantages of this system compared to other systems currently in existence, the following can be mentioned:
a. Possibility of separately analysing the resistance to inhibitors of protease and of reverse transcriptase. This makes it possible to conduct an independent evaluation of resistances to different pharmacological groups.
b. Greater efficacy in the evaluation of viral isolates with low replicative capacity.
c. It permits monitoring of certain patients in therapeutic failure.
d. With regard to the system patented by Virologic (U.S. Pat. No. 5,837,464), the system of recombinant viral clones of the present invention has notable differences, leading to the important advantages cited earlier, namely:
   Virologic clones the luciferase gene in the envelope and the applicant in Nef.
   Virologic uses similar but not identical enzymes to those used here.
   The system of the present invention has modified the NL4.3 skeleton by mutagenesis.
   In the present invention, multiple cycle vectors and separate cloning of the RT and Protease, and evaluation of the gag-Protease and gag-pol fragments can be used, aspects which the Virologic system does not permit.

### 2.2.—Determination System of the Replicative Capacity

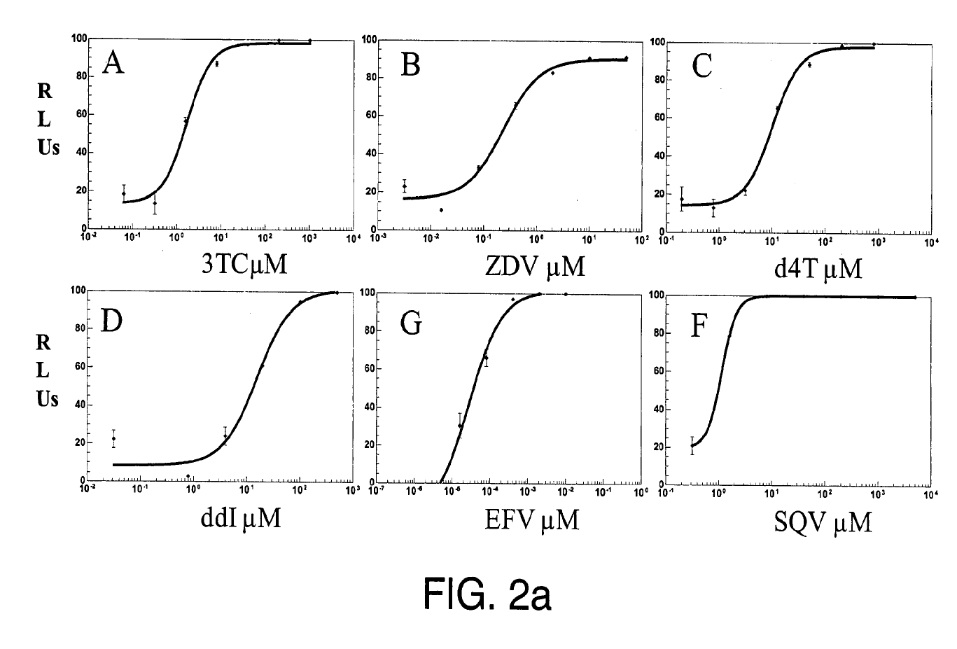

FIG. 3 represents a histogram showing the improvement in the recovery of a virus with multiple resistance mutations in the Protease and RT when separate cloning is carried out of both fragments than with the complete pol gene. This effect is due to the accumulation of loss of viral fitness which can result in viruses with low replicative capacity that are difficult to detect in single cycle tests when the loss of fitness owing to mutations in the Reverse Transcriptase and Protease are added together.

The viral clones submitted for evaluation were the following

IP HIV NL LacZ/pol Ren,

IP HIV NL LacZ/pr Ren,

IP HIV NL LacZ/rt Ren and

IP HIV NL LacZ/gag-pr Ren

The most outstanding advantages of the inventive system compared to others currently in existence are the following:
a. The system is very sensitive since it uses renilla activity.
b. The system directly measures antiviral activity, unlike the MTT test which measures protection against the cytopathic effect, which is an indirect measurement of viral replication.
c. It has the possibility of separately cloning reverse transcriptase or protease, which permits it to define in which protein the loss of replicative capacity lies.
d. It has the possibility of jointly cloning the gag-pro gene which permits a definition to be made of the role of excision sites in the polyprotein of the viral core by the protease of HIV in improving viral replicative capacity.
e. The use of viral systems in which replication can be detected with a limited number of cycles means that, when viral escape exists, the neutralisation curves in multiple cycles of the virus are not equalised.

### 2.3.—Determination System of Viral Tropism, Phenotypic Resistances to Fusion Inhibitors

The proposed invention is based on the system of cloning gene fragments of the envelope in carrier viral vectors of marker genes. A cell is required which expresses at the same time the two largest coreceptors of the virus CCR5 and CXCR4.

### (A) General Description of the Technique.

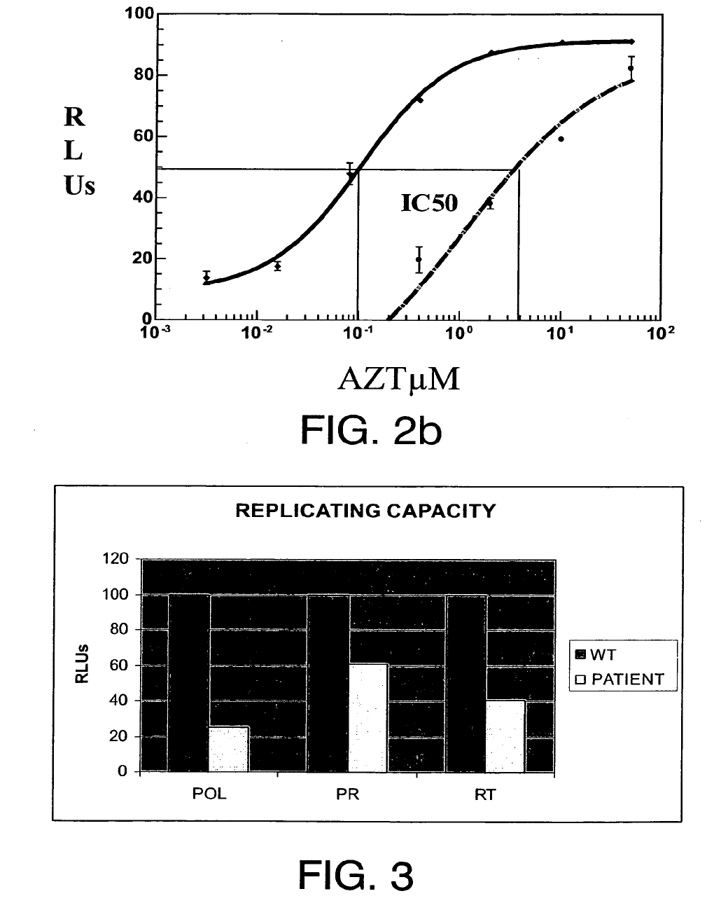

Starting from 0.5 ml of the patient's plasma, the extraction of RNA from the HIV is carried out.
The viral RNA is retrotranscribed and then amplified using primers for each viral gene by means of chain reaction of the polymerase. The primers include specific restriction sites for later cloning in the reference virus and include the entire envelope of the virus.
Following enzymatic digestion of the amplificate and of the reference virus, an in vitro ligation process is carried out using the T4 ligase.
The population of the generated recombinant provirus is transfected in the cell line 293-T and acts as a producer cell of recombinant viruses.
The infectious progeny of recombinant viruses is gathered 48 hours after the transfection and is used for infecting the cell line SSPA-B7 which expresses CCR5 and CXCR4 (FIG. 4)

### (B) Virus:

This starts from the proviral vector NL4.3

(Adachi et al. 1986). These clones have been genetically modified in the laboratory producing multiple cycle viral clones and in which the complete env gene is cloned. With the generated recombinant virus, viral tropism or the resistance of the entry to inhibitors can be analysed. The corresponding viral clones are the following:
IP HIV NL Ren,
IP HIV NL JRRen and
IP HIV NL LacZ/env Ren

### (C) Cells

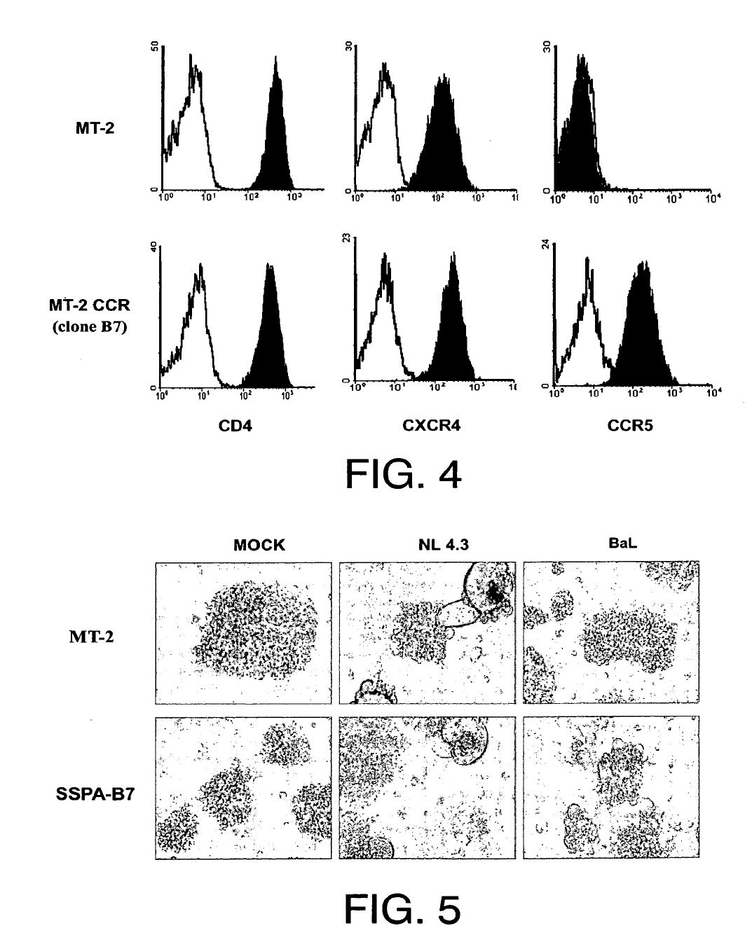

A cellular clone of SSPA-B7 has been generated by means of genetic engineering techniques which expresses the receptor CCR5 (FIG. 4) and which is susceptible to infection by the virus R5, X4 or R5X4. Infection by these three variants is productive and induces cytopathic effect (FIG. 5).
The most outstanding advantages of the inventive system compared to others currently in existence are the following:
a. The possibility of cloning the complete envelope of HIV. Other systems use recombination which presents a very low efficacy or they clone smaller fragments of the envelope.
b. The availability of a cell which expresses receptors CCR5 and CXCR4.
c. Its use in phenotypic tests on entry inhibitors.

### 2.4.—System for Detection and Titration of Neutralising Antibodies

(A) General Description of the Technique.

The proposed invention is based on the measurement of the neutralising activity in patients' serum against infection of a permissive line of marker gene carrier viruses and with different envelopes. The system includes viral clones with envelopes R5 and X4 and a cell which expresses the two largest coreceptors of the virus CCR5 and CXCR4.

(B) Virus:

This starts from the proviral vector NL4.3 (Adachi et al. 1986). These clones have been genetically modified in the laboratory producing multiple cycle viral clones in which the complete env gene is cloned. With the generated recombinant virus one can analyse the neutralising capacity against different envelopes of the virus including that of the patient's own virus. The corresponding viral clones thus obtained and evaluated were the following
IP HIV NL Ren,
IP HIV NL JRRen and
IP HIV NL LacZ/env Ren

(C) Cells

A SSPA-B7 cellular clone has been generated by means of genetic engineering techniques which expresses the receptor CCR5 (FIG. 4) and which is susceptible to infection by the virus R5, X4 or R5×4. Infection by these three variants is productive and induces cytopathic effect (FIG. 5).

(D) Results

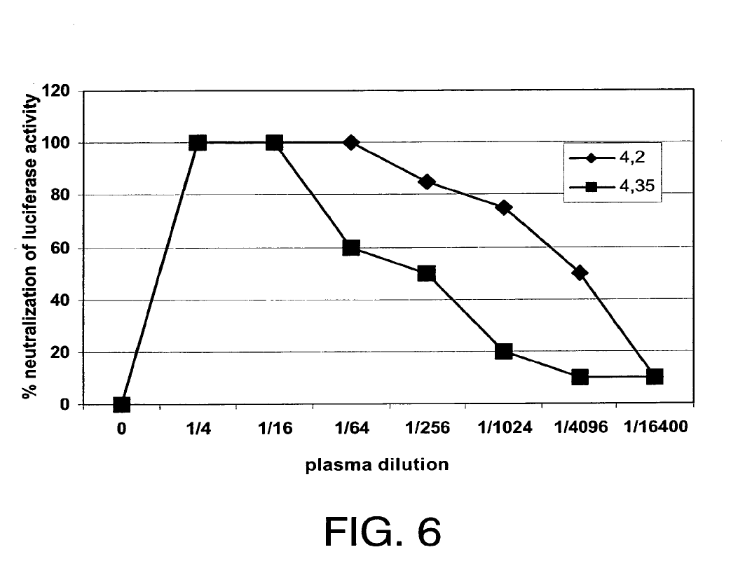

The results of the tests conducted with these viral clones according to the inventive system for the detection and titration of neutralising antibodies are illustrated in FIG. 6.

Said FIG. 6 is a graphic representation showing the results of the analysis of the neutralising capacity of HIV NL Ren virus of a patient's plasma before (4.35) and after (4.2) conducting a series of controlled treatment interruptions. In the classic MTT test, the differences between the two samples could not be observed.

Among the advantages of the system compared to others currently in existence, the following have to be highlighted:
a. The system is very sensitive since it uses renilla activity.
b. The system directly measures antiviral activity, unlike the MTT test which measures protection against the cytopathic effect, which is an indirect measurement of viral replication.
c. It has the possibility of cloning the complete envelope of different HIVs or even that of the patient himself (autologous neutralisation test).
d. The use of viral systems in which replication can be detected with a limited number of cycles means that, when viral escape exists, the neutralisation curves in multiple cycles of the virus are not equalised.
e. The availability of a cell which expresses the receptors CCR5 and CXCR4.
f. The renewed interest in studying neutralising antibodies in the context of the new vaccine models and their use as surrogate marker which will increase the demand for these tests in the immediate future.
g. The system is robotisable.

### 2.5.—System for Screening Compounds and Products Having Potential Activity Against HIV

(A) General Description of the Technique.

The proposed invention is based on the measurement of antiviral activity against HIV of chemical compounds and derivatives of natural products using marker gene carrier viruses.

(B) Virus:

This starts from the proviral vector NL4.3 (Adachi et al. 1986). These clones have been genetically modified in the laboratory producing multiple cycle viral clones with the envelope of HIV.

By limiting the infection to a single replication cycle (18 h), antiviral activity can be detected from the entry process up to the transcription/translation of viral proteins. In this period of time, antiviral action in later stages, as in the case of protease inhibitors or viral encapsidating or gemmation inhibitors, would not be detected.

For these cases, renilla activity beyond the first cycle (18 hours) is evaluated. A drop in the luciferase activity in the single and multiple cycle indicates that the compound acts in stages prior to the processing of viral proteins. Nevertheless, if it only acts on the multiple cycle, this would indicate that it acts in post-integration/viral replication stages. The corresponding recombinant viral clone is as follows:
IP HIV NL Ren

(C) Results

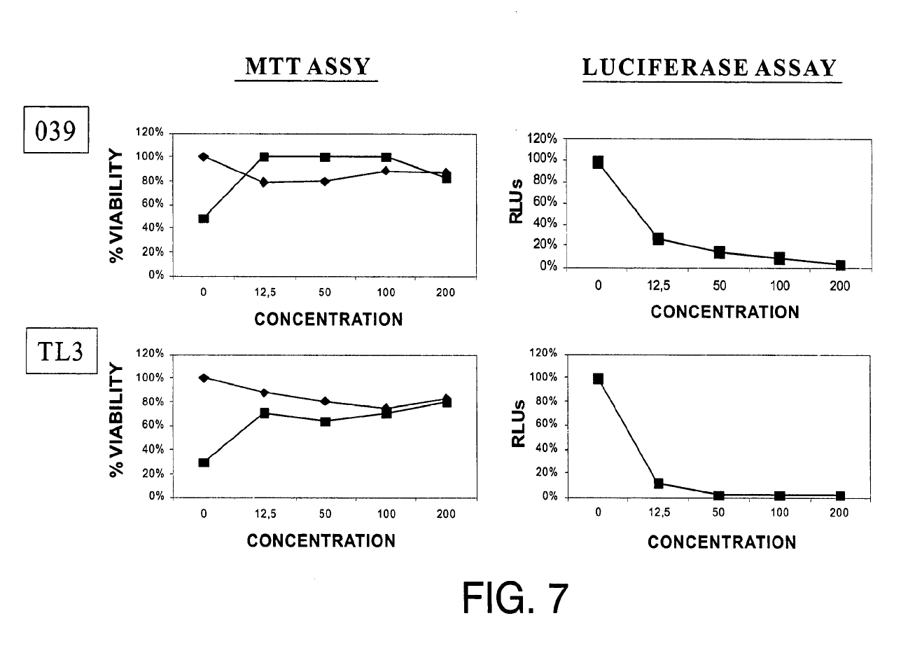

The results of the tests conducted with these viral clones according to the inventive system for the screening of compounds are illustrated in FIG. 7, where the graphic representations are shown corresponding to the analysis of antiviral activity of two compounds derived from plant products. In the classic MTT test, the toxicity of the compound (line with diamonds) and the protection against the cytopathic effect (lines with squares) are measured. The panels on the right analyse the inhibition of the replication of a luciferase virus. The mechanism of action of both compounds is being characterised at this moment and we know that compound 039 is a viral entry inhibitor.

The following advantages of the system can be highlighted compared to others currently in existence:
a. No antiviral activity evaluation systems have been described using recombinant viruses.
b. The system is very sensitive since it uses renilla activity.
c. The system directly measures antiviral activity, unlike the MTT test which measures protection against the cytopathic effect, an indirect measure of viral replication.
d. The system is robotisable and applicable to mass screening.

1. An HIV-based recombinant viral clone containing the following elements in 5′ to 3′ direction:
a. LTR or redundant terminal sequences (R) of the proviral DNA of NL4.3;
b. gag is the gene which codes the p55 capsid protein formed by the 3 protein subunits MA, CA and NC, said gag gene can comprise:
b.1) NarI restriction site at position 637 made unique by directed mutagenesis;
c. pol is the gene which codes the viral enzymes needed for the viral replication process, and whose 5′ end overlaps with gag element, if b.1) does not apply, said pol gene can comprise restriction sites generated by directed mutagenesis, said restriction sites being selected from the following group:
c.1) NcoI-AgeI restriction sites at positions 2593 and 3485 respectively,
c.2) ApaI-NcoI restriction sites at positions 2006 and 2593 respectively, and
c.3) ApaI-AgeI restriction sites at positions 2006 and 3485 respectively, or
if b.1) does apply, said pol gene can comprise:
c.4) KspI restriction site generated by directed mutagenesis at position 4498, and
c.5) ApaI-NcoI restriction sites generated by directed mutagenesis at positions 2006 and 2593 respectively;
d. vif is the gene that codes the protein Vif, it's 5′ end overlaps with pol element and it's 3′ end overlaps vpr element;
e. vpr is the gene that codes the protein Vpr and it's 5′ end overlaps vif element;
f. tat is the gene that codes the protein Tat, it's second exon is contained inside env sequence;
g. vpu is the gene that codes Vpu;
h. env is the gene which codes the protein gp160 of the viral envelope, if b.1), c.1), c.2), c.3), c.4) and c.5) do not apply, said env gene can comprise:
h.1) XbaI-NotI restriction sites generated by directed mutagenesis at positions 6114 and 8796 respectively;
i. rev is the gene that codes the protein Rev, it's second exon is contained inside env sequence;
j. NotI is a restriction site for NotI enzyme, that has been introduced by directed mutagenesis at position 8796 of the viral genome;
k. XhoI is a restriction site for the XhoI enzyme, in position 8887 of the viral genome;
l. nef is the gene that codes protein Nef, and is deleted at the bases encompassing the fragment between positions 8796 and 8887 of the viral genome by cutting with restriction enzymes NotI and XhoI;
m. Renilla is the gene that codes the luciferase reporter protein Renilla, and that has been cloned in restriction sites NotI-XhoI in position 5′ and 3′, respectively;
n. LTR, whose 5′ end overlaps with the 3′ end of nef element; and
o. LacZ gene cloned in restriction sites generated by directed mutagenesis, substituting fragments of the viral genome in order to allow further replacement of such sequences with fragments from isolates coming from patients, said restriction sites being selected from the following group:
o.1) ApaI-AgeI restriction sites, in positions 5′ and 3′, respectively, wherein c.3) applies, said LacZ gene is cloned between positions 2006 and 3485, substituting the fragment of pol gene that encode the protease and the reverse transcriptase regions of the viral genome,
o.2) ApaI-NcoI restriction sites in positions 5′ and 3′, respectively, wherein c.2) applies or b.1), c.4) and c.5) applies, said LacZ gene is cloned between positions 2006 and 2593, substituting the fragment of pol gene that encode the protease region of the viral genome,
o.3) NcoI-AgeI restriction sites in positions 5′ and 3′, respectively, wherein c.1) applies, said LacZ gene is cloned between positions 2593 and 3485, substituting the fragment of pol gene that encode the reverse transcriptase region of the viral genome, and
o.4) XbaI-NotI restriction sites in positions 5′ and 3′, respectively, wherein h.1) applies, said LacZ gene is cloned between positions 6114 and 8796, substituting the fragment of env gene of the viral genome.

2. The recombinant viral clone according to claim 1, wherein applying c.1) and o.3) defined in claim 1, said clone is the clone IP HIV NL LacZ/rt Ren, deposited in the Spanish Collection of Type Cultures as CECT 5845, which possesses a unique restriction site for enzyme NcoI that has been introduced by directed mutagenesis at the position 2593 of the DNA sequence, and the LacZ gene is cloned in NcoI-AgeI restriction sites in positions 5′ and 3′, respectively, substituting the fragment of pol gene that codes the reverse transcriptase.

3. The recombinant viral clone according to claim 1, wherein applying c.2) and o.2) defined in claim 1, said clone is the clone IP HIV NL LacZ/pr Ren, deposited in the Spanish Collection of Type Cultures as CECT 5846, which possesses a unique restriction site for NcoI enzyme introduced by directed mutagenesis in position 2593 of the DNA sequence, and LacZ gene is cloned between restriction sites ApaI-NcoI in positions 5′ and 3′, respectively, substituting the fragment of pol gene that encodes the protease.

4. The recombinant viral clone according to claim 1, wherein applying c.3) and o.1) defined in claim 1, said clone is the clone IP HIV NL LacZ/pol Ren, deposited in the Spanish Collection of Type Cultures as CECT 5847, which possesses the LacZ gene cloned between restriction sites ApaI-AgeI in positions 5′ and 3′, respectively, substituting the fragment of pol gene that encodes the protease and the reverse transcriptase.

5. The recombinant viral clone according to claim 1, wherein applying b.1), c.4), c.5) and o.2) defined in claim 1, said clone is the clone IP HIV NL LacZ/gag-pr Ren, deposited in the Spanish Collection of Type Cultures as CECT 5848, which possesses unique restriction sites for enzymes NarI and KspI, this last one introduced by directed mutagenesis, at positions 637 and 4498, respectively, in the DNA sequence, and LacZ gene is cloned between the restriction sites ApaI-NcoI in positions 5′ and 3′, respectively, substituting the fragment of gag-pol gene that encodes the protease.

6. The recombinant viral clone according to claim 1, wherein applying h.1) and o.4) defined in claim 1, said clone is the clone IP HIV NL LacZ/env Ren, deposited in the Spanish Collection of Type Cultures as CECT 5844, which possesses a unique restriction site for XbaI enzyme, introduced by directed mutagenesis in position 6114 of the DNA sequence, and LacZ gene is cloned between restriction sites XbaI-NotI in positions 5′ and 3′, respectively, substituting env gene.

7. Method of using a recombinant viral clone selected from the group consisting of: clone IP HIV NL LacZ/rt Ren (CECT 5845), clone IP HIV NL LacZ/pr Ren (CECT 5846), clone IP HIV NL LacZ/pol Ren (CECT 5847), clone IP HIV NL LacZ/gag-pr Ren (CECT 5848), and clone IP HIV NL LacZ/env Ren (CECT 5844), to determine phenotypic resistances to antiretroviral drugs for the treatment of HIV infection, comprising the following steps:
a) extraction of RNA from the HIV from the patient's plasma,
b) retrotranscription and amplification of said patient's viral RNA using specific primers for each viral gene by means of nested polymerase chain reaction, said primers including specific restriction sites generated by directed mutagenesis, said specific restriction sites being select from the following group:
b.i) NarI-KspI at positions 637 and 4498 respectively and ApaI-NcoI restriction sites at positions 2006 and 2593 respectively, for later cloning the amplificate patient's fragment of gag-pol gene between such positions replacing the LacZ gene of the recombinant viral clone IP HIV NL LacZ/gag-pr Ren (CECT 5848) defined in claim 5,
b.ii) NcoI-AgeI restriction sites at positions 2593 and 3485 respectively, for later cloning the amplificate patient's fragment of pol gene between such positions replacing the LacZ gene of the recombinant viral clone IP HIV NL LacZ/rt Ren (CECT 5845) defined in claim 2,
b.iii) ApaI-NcoI restriction sites at positions 2006 and 2593 respectively, for later cloning the amplificate patient's fragment of pol gene between such positions replacing the LacZ gene of the recombinant viral clone IP HIV NL LacZ/pr Ren (CECT 5846) defined in claim 3,
b.iv) ApaI-AgeI restriction sites at positions 2006 and 3485 respectively, for later cloning the amplificate patient's fragment of pol gene between such positions replacing the LacZ gene of the recombinant viral clone IP HIV NL LacZ/pol Ren (CECT 5847) defined in claim 4, and
b.v) XbaI-NotI restriction sites at positions 6114 and 8796 respectively, for later cloning the amplificate patient's fragment of env gene between such positions replacing the LacZ gene of the recombinant viral clone IP HIV NL LacZ/env Ren (CECT 5844) defined in claim 6,
c) enzymatic digestion of the amplificate and the recombinant viral clone and in vitro ligation process carried out using the T4 ligase for generating the recombinant viral clone carrying corresponding amplified fragment from the patient as defined in b.i) to b.v),
d) the generated recombinant viral clone described in c) is transfected in the 293T cell line,
e) the infectious progeny of the generated recombinant viral clone described in d) is gathered 48 hours after the transfection and is used for infecting the SSPA-B7 cell line,
f) determining the resistance to Protease inhibitors, Reverse Transcriptase inhibitors by evaluating the IC50 in the infection of the SSPA-B7 target cells in comparison with a recombinant viral clone selected from the group consisting of: clone IP HIV NL LacZ/rt Ren (CECT 5845), clone IP HIV NL LacZ/pr Ren (CECT 5846), clone IP HIV NL LacZ/pol Ren (CECT 5847), clone IP HIV NL LacZ/gag-pr Ren (CECT 5848), and clone IP HIV NL LacZ/env Ren (CECT 5844), without any associated resistance mutations, and
g) reading the sensitivity to said Protease inhibitors, Reverse Transcriptase inhibitors, by quantifying the capacity of the generated recombinant viral clone described in e) to complete a replication cycle which is quantified by measuring the renilla activity in the infected SSPA-B7 target cells by means of a luminometer.

8. Method of using a recombinant viral clone selected from the group consisting of: clone IP HIV NL LacZ/rt Ren (CECT 5845), clone IP HIV NL LacZ/pr Ren (CECT 5846), clone IP HIV NL LacZ/pol Ren (CECT 5847), clone IP HIV NL LacZ/gag-pr Ren (CECT 5848), and clone IP HIV NL LacZ/env Ren (CECT 5844), for determining the replicative capacity in a generated recombinant viral clone carrying a sequence fragment from a patient with HIV infection, said method comprising the following steps:
a) extraction of RNA from the HIV from the patient's plasma,
b) retrotranscription and amplification of said patient's viral RNA using specific primers for each viral gene by means of nested polymerase chain reaction, said primers including specific restriction sites generated by direct mutagenesis, said specific restriction sites being select from the following group:
b.i) NarI-KspI at positions 637 and 4498 respectively and ApaI-NcoI restriction sites at positions 2006 and 2593 respectively, for later cloning the amplificate patient's fragment of gag-pol gene between such positions replacing the LacZ gene of the recombinant viral clone IP HIV NL LacZ/gag-pr Ren (CECT 5848) defined in claim 5,
b.ii) NcoI-AgeI restriction sites at positions 2593 and 3485 respectively, for later cloning the amplificate patient's fragment of pol gene between such positions replacing the LacZ gene of the recombinant viral clone IP HIV NL LacZ/rt Ren (CECT 5845) defined in claim 2,
b.iii) ApaI-NcoI restriction sites at positions 2006 and 2593 respectively, for later cloning the amplificate patient's fragment of pol gene between such positions replacing the LacZ gene of the recombinant viral clone IP HIV NL LacZ/pr Ren (CECT 5846) defined in claim 3,
b.iv) ApaI-AgeI restriction sites at positions 2006 and 3485 respectively, for later cloning the amplificate patient's fragment of pol gene between such positions replacing the LacZ gene of the recombinant viral clone IP HIV NL LacZ/pol Ren (CECT 5847) defined in claim 4, and
b.v) XbaI-NotI restriction sites at positions 6114 and 8796 respectively, for later cloning the amplificate patient's fragment of env gene between such positions replacing the LacZ gene of the recombinant viral clone IP HIV NL LacZ/env Ren (CECT 5844) defined in claim 6,
c) enzymatic digestion of the amplificate and the recombinant viral clone, and in vitro ligation process carried out using the T4 ligase for generating the recombinant viral clone carrying corresponding amplified fragment from the patient as defined in b.i) to b.v),
d) the generated recombinant viral clone described in c) is transfected in the 293T cell line,
e) the infectious progeny of the generated recombinant viral clone described in d) is gathered 48 hours after the transfection and is used for infecting the SSPA-B7 cell line,
f) infecting said SSPA-B7 cell cultures with the cited generated recombinant viral clone described in e) or with the wild virus, and
g) measuring luciferase activity produced in the infected SSPA-B7 target infected cells described in step
f) by quantifying the capacity of the generated recombinant viral clone described in step f) to complete a replication cycle which is quantified by measuring the renilla activity by means of a luminometer.

9. Method of using the recombinant viral clone IP HIV NL LacZ/env Ren (CECT 5844) defined in claim 6, to characterise viral tropism in HIV infection, comprising the following steps:
a) extraction of RNA from the HIV from the patient's plasma,
b) retrotranscription and amplification of said patient's viral RNA using specific primers by means of nested polymerase chain reaction, said primers including XbaI-NotI restriction sites generated by direct mutagenesis at positions 6114 and 8796 respectively, for later cloning the amplified fragment of env gene between such positions replacing the LacZ gene of the recombinant viral clone defined in claim 6,
c) enzymatic digestion of the amplificate and the recombinant viral clone, and in vitro ligation process carried out using the T4 ligase for generating the recombinant virus clone carrying corresponding amplified fragment from the patient as defined in b),
d) the generated recombinant virus clone described in c) is transfected in the 293T cell line,
e) the infectious progeny of the generated recombinant viral clone defined in d) is gathered 48 hours after the transfection and is used for infecting target cells carrying either the CCR5 or the CXCR4 receptor for HIV-1, and
f) characterizing the viral tropism of the generated recombinant viral clone defined in e) by measuring the luciferase activity produced in said target cells, by quantifying the capacity of said generated recombinant viral clone to complete a replication cycle which is quantified by measuring the renilla activity by means of a luminometer.

10. Method of using the recombinant viral clone IP HIV NL LacZ/env Ren (CECT 5844) defined in claim 6, to detect neutralising antibodies against HIV, comprising the following steps:
a) obtaining serum of seropositive patients for HIV and of non-infected individuals subjected to vaccination,
b) extraction of RNA from the HIV from the patient'serum,
c) retrotranscription and amplification of said patient's viral RNA using specific primers by means of nested polymerase chain reaction, said primers including XbaI-NotI restriction sites generated by direct mutagenesis at positions 6114 and 8796 respectively, for later cloning the amplified fragment of env gene between such positions replacing the LacZ gene of the recombinant viral clone defined in claim 6,
d) enzymatic digestion of the amplificate and the recombinant viral clone, and in vitro ligation process carried out using the T4 ligase for generating the recombinant viral clone carrying corresponding amplified fragment from the patient as defined in c),
e) the generated recombinant viral clone described in d) is transfected in the 293T cell line,
f) the infectious progeny of the generated recombinant viral clone defined in e) is gathered 48 hours after the transfection and is used for infecting the SSPA-B7 cell line carrying either the CCR5 or the CXCR4 receptor for HIV-1, and
g) evaluation of the neutralising capacity of said patients serum towards the generated recombinant viral clone defined in f) by direct analyzing the viral replication and its inhibition by serial sera dilutions from said patients by measuring the luciferase activity produced in the infected SSPA-B7 target infected cells described in step f) by quantifying the capacity of the generated recombinant viral clone described in step f) to complete a replication cycle which is quantified by measuring the renilla activity by means of a luminometer.

11. Method of using a recombinant viral clone selected from the group consisting of: clone IP HIV NL LacZ/rt Ren (CECT 5845), clone IP HIV NL LacZ/pr Ren (CECT 5846), clone IP HIV NL LacZ/pol Ren (CECT 5847), clone IP HIV NL LacZ/gag-pr Ren (CECT 5848), and clone IP HIV NL LacZ/env Ren (CECT 5844), to screen and characterise compounds for antiviral activity towards HIV, comprising the following steps:
a) extraction of RNA from the HIV from the patient's plasma,
b) retrotranscription and amplification of said patient's viral RNA using specific primers for each viral gene by means of nested polymerase chain reaction, said primers including specific restriction sites generated by direct mutagenesis, said specific restriction sites being select from the following group:
b.i) NarI-KspI at positions 637 and 4498 respectively and ApaI-NcoI restriction sites at positions 2006 and 2593 respectively, for later cloning the amplificate patient's fragment of gag-pol gene between such positions replacing the LacZ gene of the recombinant viral clone IP HIV NL LacZ/gag-pr Ren (CECT 5848) defined in claim 5,
b.ii) NcoI-AgeI restriction sites at positions 2593 and 3485 respectively, for later cloning the amplificate patient's fragment of pol gene between such positions replacing the LacZ gene of the recombinant viral clone IP HIV NL LacZ/rt Ren (CECT 5845) defined in claim 2,
b.iii) ApaI-NcoI restriction sites at positions 2006 and 2593 respectively, for later cloning the amplificate patient's fragment of pol gene between such positions replacing the LacZ gene of the recombinant viral clone IP HIV NL LacZ/pr Ren (CECT 5846) defined in claim 3,
b.iv) ApaI-AgeI restriction sites at positions 2006 and 3485 respectively, for later cloning the amplificate patient's fragment of pol gene between such positions replacing the LacZ gene of the recombinant viral clone IP HIV NL LacZ/pol Ren (CECT 5847) defined in claim 4, and
b.v) XbaI-NotI restriction sites at positions 6114 and 8796 respectively, for later cloning the amplificate patient's fragment of env gene between such positions replacing the LacZ gene of the recombinant viral clone IP HIV NL LacZ/env Ren (CECT 5844) defined in claim 6,
c) enzymatic digestion of the amplificate and the recombinant viral clone, and in vitro ligation process carried out using the T4 ligase for generating the recombinant viral clone carrying corresponding amplified fragment from the patient as defined in b.i) to b.v),
d) the generated recombinant viral clone described in c) is transfected in 293T cells,
e) the infectious progeny of the generated recombinant viral clone described in d) is gathered 48 hours after the transfection and is used for infecting the SSPA-B7 cell line, in the presence of compounds with potential antiviral activity,
f) screening and characterising compounds with potential antiviral activity by analyzing the direct inhibition of HIV replication by different concentrations of a given compound through measuring the luciferase activity produced in the SSPA-B7 target infected cells defined in e) by quantifying the capacity of the generated recombinant viral clone to complete a replication cycle which is quantified by measuring the renilla activity by means of a luminometer.


SEQUENCE LISTING (24 sequences)

SEQ ID NO: 1 - DNA, 43 bp, Artificial Sequence
  Oligonucleotide
gctataagatgggtggcgcggccgcaaaaagtagtgtgattgg

SEQ ID NO: 2 - DNA, 43 bp, Artificial Sequence
  Oligonucleotide
ccaatcacactactttttgcggccgcgccacccatcttatagc

SEQ ID NO: 3 - DNA, 37 bp, Artificial Sequence
  Oligonucleotide
ccagtaaaattaaagccagccatggatggcccaaaag

SEQ ID NO: 4 - DNA, 37 bp, Artificial Sequence
  Oligonucleotide
cttttgggccatccatggctggctttaattttactgg

SEQ ID NO: 5 - DNA, 38 bp, Artificial Sequence
  Oligonucleotide
gaagcagaagtaattcccgcggagacagggcaagaaac

SEQ ID NO: 6 - DNA, 38 bp, Artificial Sequence
  Oligonucleotide
gtttcttgccctgtctccgcgggaattacttctgcttc

SEQ ID NO: 7 - DNA, 36 bp, Artificial Sequence
  Oligonucleotide
gaaaataccgcatcaggacccattcgccattcaggc

SEQ ID NO: 8 - DNA, 36 bp, Artificial Sequence
  Oligonucleotide
gcctgaatggcgaatgggtcctgatgcggtattttc

SEQ ID NO: 9 - DNA, 55 bp, Artificial Sequence
  Oligonucleotide
gcattagtagtagcaataataatagctctagagctgtggtccatagtaatcatag

SEQ ID NO: 10 - DNA, 55 bp, Artificial Sequence
  Oligonucleotide
ctatgattactatggaccacagctctagagctattattattgctactactaatgc

SEQ ID NO: 11 - DNA, 25 bp, Artificial Sequence
  Oligonucleotide
gccaaaaattgcagggcccctagga

SEQ ID NO: 12 - DNA, 35 bp, Artificial Sequence
  Oligonucleotide
tcttttgatgggtcataatacactccatgtaccgg

SEQ ID NO: 13 - DNA, 25 bp, Artificial Sequence
  Oligonucleotide
gccaaaaattgcagggcccctagga

SEQ ID NO: 14 - DNA, 36 bp, Artificial Sequence
  Oligonucleotide
catgccatggctggctttaattttactggtacagtc

SEQ ID NO: 15 - DNA, 35 bp, Artificial Sequence
  Oligonucleotide
catgccatggatggcccaaaagttaaacaatggcc

SEQ ID NO: 16 - DNA, 35 bp, Artificial Sequence; identical to SEQ ID NO: 12
  Oligonucleotide

SEQ ID NO: 17 - DNA, 30 bp, Artificial Sequence
  Oligonucleotide
ggaaaatctctagcagtggcgcccgaacag

SEQ ID NO: 18 - DNA, 36 bp, Artificial Sequence; identical to SEQ ID NO: 14
  Oligonucleotide

SEQ ID NO: 19 - DNA, 30 bp, Artificial Sequence; identical to SEQ ID NO: 17
  Oligonucleotide

SEQ ID NO: 20 - DNA, 31 bp, Artificial Sequence
  Oligonucleotide
cttgccctgtctctgctggaattacttctgc

SEQ ID NO: 21 - DNA, 25 bp, Artificial Sequence
  Oligonucleotide
tatgaaacttacggggatacttggg

SEQ ID NO: 22 - DNA, 27 bp, Artificial Sequence
  Oligonucleotide
ctgccaatcagggaagtagccttgtgt

SEQ ID NO: 23 - DNA, 42 bp, Artificial Sequence
  Oligonucleotide
gtagcaataataatagctctagagctgtggtccatagtaatc

SEQ ID NO: 24 - DNA, 33 bp, Artificial Sequence
  Oligonucleotide
tactttttgcggccgcgccacccatcttatagc